(19) United States
(12) Patent Application Publication
(10) Pub. No.: US 2017/0360840 A1
(43) Pub. Date: Dec. 21, 2017

(54) EXTRACELLULAR VESICLES WITH ENHANCED POTENCY

(71) Applicant: United Therapeutics Corporation, Silver Spring, MD (US)

(72) Inventors: Sarah HOGAN, Raleigh, NC (US); Roger Marquez ILAGAN, Durham, NC (US); Maria Pia RODRIGUEZ, Durham, NC (US); Carolina MEDRANO, Durham, NC (US); John CHEADLE, Durham, NC (US)

(73) Assignee: United Therapeutics Corporation, Silver Spring, MD (US)

(21) Appl. No.: 15/625,961

(22) Filed: Jun. 16, 2017

Related U.S. Application Data
(60) Provisional application No. 62/351,627, filed on Jun. 17, 2016.

(51) Int. Cl. A61K 35/28; C12N 15/113; A61K 38/46; C12N 9/12; C12N 5/0775; C12N 9/14; A61K 38/45

(52) U.S. Cl. CPC A61K 35/28; C12Y 306/01003; C12Y 207/0104; C12N 9/14; C12N 15/113; A61K 38/45; C12N 9/1205; C12N 5/0662; A61K 38/46; C12N 2310/141

(57) ABSTRACT

Provided are methods for isolating potent extracellular vesicle or exosome populations from mesenchymal stromal cells, and the use of the isolated extracellular vesicles or exosomes in treating vasculopathy, including pulmonary hypertension, bronchopulmonary dysplasia, and disease and conditions associated with mitochondrial dysfunction.

### CROSS REFERENCE TO RELATED APPLICATIONS

This application claims priority to U.S. Provisional Patent Application No. 62/351,627 filed Jun. 17, 2016, which is hereby incorporated by reference in its entirety.

### BACKGROUND

The present application relates to methods of isolating potent extracellular vesicles, including exosomes, and the use of extracellular vesicles or exosomes in treatment of pulmonary hypertension, including pulmonary arterial hypertension (PAH), and conditions and diseases associated with mitochondrial dysfunction.

Pulmonary hypertension is a progressive and often fatal disease characterized by increased pressure in the pulmonary vasculature. An increasing constriction of the pulmonary circulation leads to increased stress on the right heart, which may develop into right heart failure. By definition, the mean pulmonary arterial pressure (mPAP) in a case of chronic pulmonary hypertension is >25 mmHg at rest or >30 mmHg during exertion (normal value <20 mmHg). For example, pulmonary arterial hypertension, untreated, leads to death on average within 2.8 to 5 years after being diagnosed (Keily et al. (2013) BMJ346:f2028). The pathophysiology of pulmonary arterial hypertension is characterized by vasoconstriction and remodeling of the pulmonary vessels. In chronic PAH there is neomuscularization of initially unmuscularized pulmonary vessels, and the vascular muscles of the already muscularized vessels increase in circumference. This resulting increase in pulmonary arterial pressures results in progressive stress on the right heart, which leads to a reduced output from the right heart and eventually ends in right heart failure (M. Humbert et al., J. Am. Coll. Cardiol. 2004, 43, 13 S-24S). PAH is a rare disorder, with a prevalence of 1-2 per million. The average age of the patients has been estimated to be 36 years, and only 10% of the patients were over 60 years of age. Distinctly more women than men are affected (G. E. D′Alonzo et al., Ann. Intern. Med. 1991, 115, 343-349).

Numerous mechanisms have been implicated in the pathogenesis of PAH. Importantly, a suppression of global metabolism has been described downstream of aberrant mitochondrial glucose oxidation in this disease. Diminished mitochondrial function could unify many apparently unrelated abnormalities in PAH, such as the involvement of multiple cell types, the cancer-like proliferation of pulmonary vascular cells, and resistance of these cells to apoptosis. Despite evidence supporting the role of mitochondrial dysfunction in PAH, therapeutic targeting of mitochondrial function has proven difficult.

Thus, a need exists to develop improved therapeutic compositions and methods for treating pulmonary hypertension, such as by targeting mitochondrial function.

### SUMMARY

In one aspect, the present disclosure provides a method of treating (including preventing) pulmonary hypertension, comprising administering to a subject in need thereof isolated extracellular vesicles or exosomes obtained from mesenchymal stromal cells, wherein the isolated extracellular vesicles or exosomes comprise extracellular vesicles or exosomes having increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells. In some embodiments, the extracellular vesicles or exosomes comprise at least 10%, 20%, 30%, 50%, or 100% more expression of the expression products compared to the average level of the same expression product in all extracellular vesicles or exosomes obtained from the mesenchymal stromal cells.

In some embodiments, the isolated extracellular vesicles or exosomes have increased expression of protein(s) of one or more genes selected from the group consisting of (a) genes in the glycolysis pathway, (b) genes in the TCA cycle, and (c) genes in the electron transport chain. In some embodiments, the isolated extracellular vesicles or exosomes have increased expression of RNA(s) of one or more genes selected from the group consisting of (a) genes in the glycolysis pathway, (b) genes in the TCA cycle, (c) genes in the electron transport chain.

In some embodiments, (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

In some embodiments, the gene is PK. In some other embodiments, the gene is ATPase.

In some embodiments, the isolated extracellular vesicles or exosomes normalizes glucose oxidation in the subject. In some embodiments, the isolated extracellular vesicles or exosomes normalize glucose oxidation in lung tissue of the subject. In some embodiments, the isolated extracellular vesicles or exosomes has a PK activity of at least 0.15 nmol/min/mL.

In some embodiments, isolated extracellular vesicles or exosomes are capable of reducing Right Ventricular Systolic Pressure (RVSP) of mice subjected to a three-week chronic hypoxia exposure by at least 10%, compared to control mice subjected to a three-week chronic hypoxia exposure and treated with PBS.

In some embodiments, the isolated extracellular vesicles or exosomes are capable of increasing O₂ consumption by smooth muscle cell (SMC) cell lysates subjected to a 24-hour hypoxia exposure by at least 20% compared to control SMC cell lysates subjected to a 24-hour hypoxia exposure and treated with PBS control.

In another aspect, the present disclosure provides a method of treating a disease or condition associated with mitochondrial dysfunction, comprising administering to a subject in need thereof isolated extracellular vesicles or exosomes obtained from mesenchymal stromal cells, wherein the isolated extracellular vesicles or exosomes comprise extracellular vesicles or exosomes having increased expression of one or more expression products selected from the group consisting of (a) genes in the glycolysis pathway, (b) genes in the TCA cycle, and (c) genes in the electron transport chain as compared to the average level of the expression product in all extracellular vesicles or exosomes obtained from the mesenchymal stromal cells.

In some embodiments, the isolated extracellular vesicles or exosomes have increased expression of protein(s) of one or more genes selected from the group consisting of (a) genes in the glycolysis pathway, (b) genes in the TCA cycle, and (c) genes in the electron transport chain. In some embodiments, the isolated extracellular vesicles or exosomes have increased expression of RNA(s) of one or more genes selected from the group consisting of (a) genes in the glycolysis pathway, (b) genes in the TCA cycle, and (c) genes in the electron transport chain.

In some embodiment, (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

In some embodiments, the gene is PK. In some other embodiments, the gene is ATPase. In some embodiments, the isolated extracellular vesicles or exosomes normalize glucose oxidation in lung tissue of the subject. In some embodiments, the isolated extracellular vesicles or exosomes have a PK activity of at least 0.15 nmol/min/mL.

In some embodiments, the disease or condition associated with mitochondrial dysfunction is associated with decreased mitochondrial glucose oxidation in the subject. In some embodiments, the disease or condition associated with mitochondrial dysfunction is selected from the group consisting of Friedreich's ataxia, Leber's Hereditary Optic Neuropathy, Kearns-Sayre Syndrome, Mitochondrial Encephalomyopathy with Lactic Acidosis and Stroke-Like Episodes, Leigh syndrome, obesity, atherosclerosis, amyotrophic lateral sclerosis, Parkinson's Disease, cancer, heart failure, myocardial infarction (MI), Alzheimer's Disease, Huntington's Disease, schizophrenia, bipolar disorder, fragile X syndrome, and chronic fatigue syndrome.

In another aspect, the present disclosure provides a method of isolating extracellular vesicles or exosomes capable of treating or preventing pulmonary hypertension, comprising the following steps: (a) providing a culture media of mesenchymal stromal cells comprising extracellular vesicles or exosomes; (b) separating at least a portion of the extracellular vesicles or exosomes from the other components of the culture media; and (c) isolating an extracellular vesicle or exosome population from other extracellular vesicle or exosome populations, wherein the population has increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells.

In another aspect, the present disclosure provides a method of isolating extracellular vesicles or exosomes capable of treating or preventing pulmonary hypertension, comprising the following steps: (a) providing a culture media of mesenchymal stromal cells comprising extracellular vesicles or exosomes; (b) separating at least a portion of the extracellular vesicles or exosomes from the other components of the culture media; (c) separating different populations of extracellular vesicles or exosomes based on molecular size; (d) treating hypoxia-exposed mice with the different populations of extracellular vesicles or exosomes; (e) measuring Right Ventricular Systolic Pressure (RVSP) of normoxia mice, hypoxia-exposed mice and hypoxia exposed mice treated with the extracellular vesicles or exosomes; and (f) identifying a potent population of extracellular vesicles or exosomes based on the RVSP.

In some embodiments, a population of extracellular vesicles or exosomes is potent if the ratio of RVSP of hypoxia-exposed mice treated with the extracellular vesicles or exosomes to RVSP of hypoxia-exposed mice is 0.85 or less.

In some embodiments, a population of extracellular vesicles or exosomes is potent if delta RVSP is less than 5 mmHg, wherein delta RVSP is RVSP of hypoxia-exposed mice treated with extracellular vesicles or exosomes minus RVSP of normoxia mice.

In some embodiments, in step c, different populations of extracellular vesicles or exosomes are separated by phospholipid detection.

In some embodiments, the potent population of extracellular vesicles or exosomes have increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells. In some embodiments, the potent population of extracellular vesicles or exosomes have increased expression of protein(s) of one or more genes selected from the group consisting of (a) genes in the glycolysis pathway, (b) genes in the TCA cycle, and (c) genes in the electron transport chain. In some embodiments, the potent population of extracellular vesicles or exosomes have increased expression of RNA(s) of one or more genes selected from the group consisting of (a) genes in the glycolysis pathway, (b) genes in the TCA cycle, and (c) genes in the electron transport chain.

In some embodiments, (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

In some embodiments, the gene is PK. In some embodiments, the gene is ATPase.

In another aspect, the present disclosure provides a method of isolating extracellular vesicles or exosomes capable of treating or preventing bronchopulmonary dysplasia, comprising the following steps: (a) providing a culture media of mesenchymal stromal cells comprising extracellular vesicles or exosomes; (b) separating at least a portion of the extracellular vesicles or exosomes from the other components of the culture media; (c) separating different populations of extracellular vesicles or exosomes based on molecular size; (d) treating hypoxia-exposed mice with the different populations of extracellular vesicles or exosomes; (e) measuring Right Ventricular Systolic Pressure (RVSP) of normoxia mice, hypoxia-exposed mice and hypoxia exposed mice treated with the extracellular vesicles or exosomes; and (f) identifying a potent population of extracellular vesicles or exosomes based on the RVSP.

In some embodiments, step (b) of the method separates a portion of the extracellular vesicles or exosomes from the other components of the culture media by size exclusion chromatography.

In another aspect, the present disclosure provides a composition comprising isolated extracellular vesicles or exosomes obtained according to any of the methods described in this disclosure. In some embodiments, the isolated extracellular vesicles or exosomes have a mean diameter of about 100 nm. In some embodiments, the isolated extracellular vesicles or exosomes express FLOT and/or ANXA2. In some embodiments, the isolated extracellular vesicles or exosomes have increased expression of mir204, compared to the average amount of mir204 in all extracellular vesicles or exosomes of the mesenchymal stromal cells. In some embodiments, the isolated extracellular vesicles or exosomes have are secreted from MSCs containing increased expression of CD105, GAPDH, DLST, and/or ATP5A1, compared to the average amount of CD105, GAPDH, DLST, and/or ATP5A1 in all extracellular vesicles or exosomes of the mesenchymal stromal cells. In some embodiments, the isolated extracellular vesicles or exosomes have increased RNA expression of SORCS1, FHIT and/or ANKRD30BL, compared to the average amount of SORCS1, FHIT and/or ANKRD30BL in all extracellular vesicles or exosomes of the mesenchymal stromal cells. In some embodiments, the isolated extracellular vesicles or exosomes are substantially free of MHCII contaminants. In some embodiments, the isolated extracellular vesicles or exosomes are substantially free of fibronectin.

In another aspect, the present disclosure provides a method of treating or preventing bronchopulmonary dysplasia, comprising administering to a subject in need thereof isolated extracellular vesicles or exosomes obtained according any of the methods described in this disclosure. In some embodiments, the isolated extracellular vesicles or exosomes increase immunomodulatory capacity of the subject. In some embodiments, the isolated extracellular vesicles or exosomes reduces IL-6 and/or TNFα expression in the subject. In some embodiments, the isolated extracellular vesicles or exosomes promote angiogenesis of the subject. In some embodiments, the isolated extracellular vesicles or exosomes reduce hyperoxia-induced apoptosis in the subject. In some embodiments, the isolated extracellular vesicles or exosomes reduces Cytochrome C level in the subject. In some embodiments, the isolated extracellular vesicles or exosomes increase mitochondrial metabolism of the subject. In some embodiments, the isolated extracellular vesicles or exosomes restore tube formation in the subject. In some embodiments, the isolated extracellular vesicles or exosomes upregulate GLUD1 and/or PDH gene expression in the subject. In some embodiments, the isolated extracellular vesicles or exosomes downregulate PDK4 gene expression in the subject. In some embodiments, the isolated extracellular vesicles or exosomes downregulate SIRT4 gene expression in the subject.

In some embodiments, the method of treating or preventing pulmonary hypertension and/or bronchopulmonary dysplasia further comprises administering sildenafil to the subject.

### BRIEF DESCRIPTION OF THE DRAWINGS

The provided drawings exemplify, but do not limit, the disclosed subject matter.

FIGS. 1A-1C show isolation of exosomes from MSC culture media and their potency in preventing chronic hypoxia-induced PAH in mice. FIG. 1A shows different exosome populations isolated based on size exclusion chromatography. FIG. 1B shows overlay of exosomes isolated by phospholipid concentration detection and A280 chromatogram. FIG. 1C shows the effect of isolated exosomes in treating mice with hypoxia-induced PAH. The potent exosome population (EXM2 shown as green dot) prevented chronic hypoxia-induced PAH in mice, while the other exosome population (EXM1 shown as red dot) did not.

FIGS. 2A-2C depict identification of potent exosome populations. FIG. 2A shows pyruvate kinase activity of the exosome populations plotted against RVSP fold change between hypoxia treatment group and hypoxia control. FIG. 2B shows pyruvate kinase activity of the exosome populations plotted against delta RVSP. The red dots represent potent exosome populations that induced a significant improvement in RVSP. The blue dots represent exosome populations that are not potent in treating hypoxia induced PAH mice. FIG. 2C shows pyruvate kinase activity of the potent exosome populations graphed in a box and whisker plot.

FIGS. 3A-3B illustrate proteomic analysis of different exosome populations. FIG. 3A shows that in potent exosome populations, proteins involved in glycolysis, the TCA cycle, and the electron transport chain that have increased expression level. FIG. 3B shows Western blot analysis of pyruvate kinase of both the potent exosome population (EXM2) and the non-potent exosome population (EXM1).

FIG. 4 shows RNAseq analysis of different exosome populations. The results demonstrated that potent exosome populations contained an enrichment of genes involved in glycolysis, TCA cycle and electron transport chain, which suggests the potential for potent exosome population for genetic reprogramming to increase glucose oxidation (a).

FIGS. 5A-5B depict O₂ consumption assay in exosome treated SMC cell lysate after 24 hour exposure to either normoxia or 4% O₂ hypoxia. FIG. 5A shows O₂ consumption calculated as slope normalized to PBS control. FIG. 5B shows O₂ consumption calculated as area under the curve normalized to PBS control. The results of FIGS. 5A and 5B show that exosomes do not significantly change the cellular O₂ consumption in normoxia-exposed SMCs. On the other hand, exosome treatment induces an increase in O₂ consumption under hypoxia stress, which indicates an increase in mitochondrial function.

FIGS. 6A-6C illustrate results of microarray analysis of SMC lysates after acute hypoxia exposure. FIG. 6A shows an increase in ENO1 gene expression in glycolysis after hypoxia exposure, which is normalized by exosome treatment. FIG. 6B shows an increase in PDHB and ACLY gene expression in the TCA cycle after hypoxia exposure, which is normalized by exosome treatment. FIG. 6C shows an increase in NDUFAF3, ATP2B4, ATP5H, ATP91 gene expression in electron transport chain after hypoxia exposure, which is normalized by exosome treatment.

FIGS. 7A-7D show metabolomics analysis of SMC lysates after acute hypoxia exposure. FIG. 7A shows fold change of metabolites levels in glycolysis. FIG. 7B shows fold change of metabolites levels in the TCA cycle. FIG. 7C shows fold change of metabolites levels in electron transport chain. FIG. 7D comparison of ATP production in live SMCs exposed to hypoxia with and without treatment with exosomes. (# means p≦0.05 compared to normoxia; $ means≦0.05 compared to hypoxia; * means p≦0.1 compared to normoxia, and p≦0.1 compared to hypoxia) The results showed a buildup of metabolites in glycolysis, TCA cycle and energy metabolism after hypoxia exposure, due to decreased flux through these pathways. Exosome treatment increased flux through these pathways, which indicates that exosome treatment increases glucose oxidation and normalizes SMC stress response to acute hypoxia.

FIGS. 8A-8E show analysis of SMC lysate after exposure to hypoxia. FIG. 8A shows activity of media LDH in SMC lysate after exposure to 24 hour hypoxia. FIG. 8B shows the level of media citrate in SMC lysate after exposure to 24 hour hypoxia. FIG. 8C shows activity of media LDH in SMC lysate after exposure to chronic 2 week hypoxia. FIG. 8D shows the level of media citrate in SMC lysate exposure to chronic 2 week hypoxia. FIG. 8E shows protein expression of pyruvate dehydrogenase active subunit (PDH E1α), the PDH E1α inhibitor pyruvate dehydrogenase kinase (PDK), and ATPase in SMC lysate exposure to chronic 2 week hypoxia. (# means p≦0.05 compared to normoxia; $ means p≦0.05 compared to hypoxia; * means p≦0.1 compared to normoxia, and p≦0.1 compared to hypoxia) The results show that exosome treatment normalizes mitochondrial function after acute and chronic hypoxia exposure.

FIG. 9 shows a proposed model for exosome treatment. In particular, Pulmonary hypertension suppresses mitochondrial glucose oxidation, leads to a decrease in global mitochondrial function. Exosome treatment normalizes glucose oxidation and improve mitochondrial capacity both by acute protein integration and/or chronic genetic upregulation of enzymes in glycolysis (1) and the TCA cycle and the election transport chain within the mitochondria (2).

FIG. 10 shows comparison of chromatograms of exosomes produced without a diafiltration step (blue) and with a diafiltration step (orange). The blue chromatogram has a much higher A280 reading than the orange one, suggesting higher amount of protein in the sample compared to the diafiltrated sample. The diafiltration step is similar to buffer exchange, which includes adding PBS buffer into the reservoir to maintain the volume while continuing to run the pump to the TFF cassette filter, once a desired concentration of exosomes is reached. Gradually, the PBS will replace the conditioned media. In order to achieve as complete of an exchange as possible, 7 total volume diafiltrations were performed to with the retentate. This step helps to remove some of the impurities in the retentate, without affecting exosomes. The presence of exosomes was verified with FLOT-1 western blots. The figure shows that the diafiltration step helps to remove impurities as shown by decreased amount of total protein and phospholipid while retaining exosomes.

FIGS. 11A-11B shows exosome production analysis. FIG. 11A shows FLOT-1 western blot analysis of 12 samples. FIG. 11B shows the size distribution of exosomes, with has a median diameter of about 100 nm.

FIG. 12 shows supplemental analysis and assessment of exemplary exosome production, including chromatogram, NTA, FLOT-1 western blot and ANXA2 western blot.

FIGS. 13A-13C shows analysis of potent exosomes. FIG. 13A shows chromatograms of COM1 (large and non-potent population of exosomes) and COM2 (small and potent population of exosomes). FIG. 13B shows plotting total protein against RVSP (in an animal model of hypoxia-induced pulmonary hypertension) from each preparation compared to hypoxia control. The results show that when more protein resides in the large non-potent fraction, the resulting preparations are potent. FIG. 13C shows plotting total protein against days fed prior to starve. The results show that positive delta protein (designating potent preparations) were all consistently fed 1 day prior to starve.

FIG. 14 shows plotting mir204 (a small non-coding RNA molecule containing about 22 nucleotides functions in RNA silencing and post-transcriptional regulation of gene expression). COM1 has higher cycle threshold (CT) value, corresponding to lower miRNA and lower potency. In contrast, COM2 has increased mir204 expression.

FIGS. 15A-15D shows that the potency of exosomes is positively correlated to CD105 protein expression (FIG. 15A), GAPDH gene expression (FIG. 15B), DLST gene expression (FIG. 15C), and ATP5A1 gene expression (FIG. 15D) in the parent MSC cell. The results show that cell metabolic health directly correlates with exosome potency.

FIGS. 16A-16B shows disrupted lung development of Bronchopulmonary Dysplasia (BPD) patients. FIG. 16A shows radiographic images of different stages of BPD. FIG. 16B shows alveolar injury in mouse models.

FIG. 17 shows hyperoxic in vitro model as well as unexisome-mediated inflammatory suppression. Alveolar cells were seeded for 24 hours, and switched to 0.1% FBS media and primed with a potent population of exosomes (unexisome) in normoxic incubator for 3 hours, and plated in hyperoxic incubation chamber for 48 hours.

FIG. 18 shows that a potent population of exosomes (unexisome) increases immunomodulatory capacity based on decreased IL-6 expression in cells exposed to hyperoxic stress (hyperoxia causes IL-6 release). “#” Denotes significance of exosome treatment compared to normoxia control. “*” Denotes significance of exosome treatment compared to hyperoxia control.

FIG. 19 shows that a potent population of exosomes (unexisome) increases immunomodulatory capacity based on reduced TNFα expression in cells exposed to hyperoxic stress (hyperoxia causes TNFα release). “*” Denotes significance of exosome treatment compared to hyperoxia control.

FIG. 20 shows that a potent population of exosomes (unexisome) have anti-apoptosis effect under hyperoxia, as indicated by increased absorbance, corresponding to increased number of cells. “*” Denotes significance of exosome treatment compared to hyperoxia control.

FIG. 21 shows that a potent population of exosomes (unexisome) reduces cytochrome C release from cells exposed to hyperoxic stress, corresponding to decreased cellular apoptosis. * Denotes significance of exosome treatment compared to hyperoxia control.

FIG. 22A shows fluorescent image of normoxia cells, cells exposed to hyperoxic stress, and cells treated with potent exosomes. FIG. 22B shows that exosomes (COM2) treatment restore tube formation in hyperoxia.

FIG. 23 shows schematics of smooth muscle cells (SMC) chronic hypoxia model. SMC switch to a proliferative, non-apoptotic phenotype in PAH and hypoxia, leads to thickening of the vessels and arteries in the lungs, causing higher pressures and ultimately damages to the heart/negative symptoms in PAH. SMCs were cultured at normoxia, 4% oxygen, and 4% oxygen with exosomes. During the culturing period, the cells were treated twice a week for two weeks with potent exosomes. The resulting SMCs were analyzed by microarray gene expression (IPA) and/or global metabolomics.

FIG. 24 shows that exosomes upregulate amino acid metabolism in chronic hypoxia by global metabolite analysis of the intermediate metabolites within pathway.

FIG. 25 shows that exosomes upregulate pyruvate and glutamate metabolism in chronic hypoxia.

FIG. 26 shows that exosomes upregulate GLUD1 gene expression in chronic hypoxia.

FIG. 27 shows that exosomes downregulate PDK4 in chronic hypoxia.

FIG. 28 shows that exosomes downregulate SIRT4 in chronic hypoxia. SIRT4 gene inhibits 2 metabolic enzymes, GLUD1 and PDH, in an in vitro PAH model. SIRT4 gene is downregulated by exosome treatment in vitro PAH model, while GLUD1 and PDH are upregulated by exosome treatment. SIRT4 is believed to be a target for exosome treatment.

FIG. 29 shows that exosomes restore TCA cycle function by upregulating the downregulated genes in hypoxia (6 out of 9 enzymes in the TCA cycle are downregulated).

FIG. 30 shows the relationship between SIRT4 and exosomes treatment.

FIG. 31 shows proposed mechanism of potent exosomes in restoring TCA cycle. In particular, hypoxia inhibits TCA cycle function by (1) upregulating SIRT4 and PDK4, which both inhibit PDH and therefore pyruvate entry into the TCA cycle (2) upregulating SIRT4, which inhibits GLUD1 and therefore glutamine entry into the TCA cycle. Potent exosomes (unexisomes) decrease both SIRT4 and PDK4, thereby increasing glutamine and pyruvate flux into the TCA cycle.

FIG. 32 shows isolation of the potent population of exosomes (COM2) using size exclusion chromatography, compared to ultra-centrifugation or gradient separation which isolate not only exosomes of non-ideal size but also protein and non-potent microvesicles contaminants.

FIG. 33 shows TEM imaging of exosomes. The results show that the isolated potent exosomes have more homogenous size and clearer image compared to ultracentrifuged samples.

FIG. 34 shows that the isolated potent exosomes are free of MHCII contamination. In contrast, commercially available exosomes from ZenBio contain MHCII contamination.

FIGS. 35A-35B show that the isolated potent exosomes are free of fibronectin and other protein contaminations based on ponceau staining. FIG. 35A shows that the isolated exosomes are free of contaminating protein fibronectin, while ZenBio commercially available ultracentrifuged preparation have fibronectin contamination. FIG. 35B shows that the exosome isolated by ultracentrifugation contained fibronectin contamination as did concentrated cell culture media. The exosomes isolated by size exclusion chromatograph are free of fibronectin contaminant as well as other protein contaminant capable of being stained by ponceau.

FIG. 36 shows RNAseq clustering analysis of the potent population and contaminant microvesicles. The results show that that the potent exosomes (COM2) are differentially clustering from contaminating microvesicles. They are enriched in SORCS1, FHIT and ANKRD30 BL.

FIG. 37 shows GAPDH expression in potent exosomes (COM2) versus non-potent exosomes (COM1). The results show that potent exosomes have a lower CT value, corresponding to higher expression level of GAPDH.

FIG. 38 shows the procedure of in vivo study of treating BPD mouse model with potent exosomes (Unexisome).

FIGS. 39A-39C show characterization of exosomes used in the in vivo study. FIG. 39A shows the concentration of exosomes is 1.2×10⁸ particles/mL. FIG. 39B shows that FLOT1 is present in exosomes based on Western blot analysis. FIG. 39C shows representative TEM imaging of exosomes.

FIGS. 40A-40B show that exosomes rescued BPD-associated alveolar simplification. FIG. 40A shows that treatment with potent exosomes rescues the hyperoxia (HYRX)-mediated increase in alveolar simplification compared to normoxia (NRMX). FIG. 40B shows quantification of mean liner intercept, which represents a surrogate of average air space diameter.

FIG. 41 shows the procedure for in vivo study of treating BPD-induced PAH mouse model of PAH secondary to BPD with potent exosomes (Unexisome).

FIGS. 42A-42C show that exosomes rescued chronic alveolar simplification. FIG. 42A shows the images of cells under normoxia, hypoxia, and cells treated with Wharton's jelly derived exosomes, and bone marrow derived exosomes. FIG. 42B shows that exosomes rescued alveolar simplification in BPD-associated PAH mouse model. FIG. 42C shows that exosomes reduced modest lung fibrosis as shown by collagen deposition in the septal area in BPD-associated PAH mouse model.

FIGS. 43A-43D show that exosomes rescued PAH pulmonary vascular remodeling demonstrated by a-smooth muscle actin stain (A and B) and pressure changes associated with PAH (C). PV-loops demonstrate a significant shift in hyperoxia (HYRX) mice, indicative of emphysema-like features of lung disease and air trapping when compared to normoxia (NRMX) controls. Exosomes showed a significant rescue in this shift, indicative of improved lung function.

FIG. 44A shows the procedure for in vivo study of treating hypoxia-induced PAH mouse model.

FIG. 44B shows that MSC exosomes prevent PAH in mice.

FIG. 45A shows the procedure of in vivo study of treating Sugen (VEGF receptor agonist) and hypoxia-induced PAH mouse model with a combination of exosomes and sildenafil. FIG. 45B shows that the combination therapy of exosome and sildenafil reversed PAH in mouse model.

FIG. 46 shows the procedure for identification of exosome-mediated mechanism of action in PAH.

FIG. 47 shows that exosomes upregulate amino acid metabolism in Sugen/hypoxia model of PAH.

### DETAILED DESCRIPTIONS

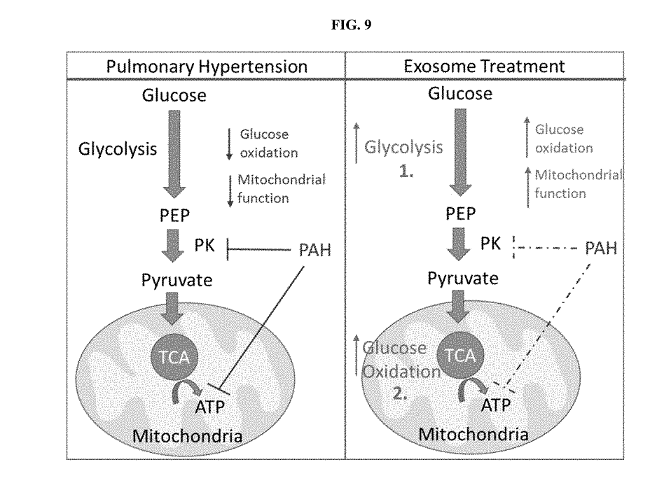

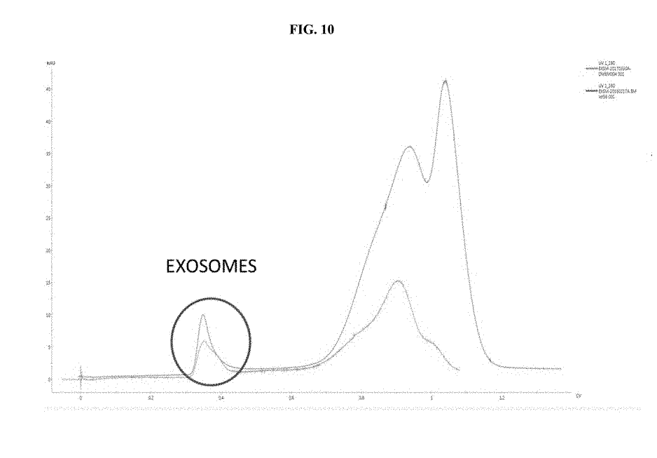

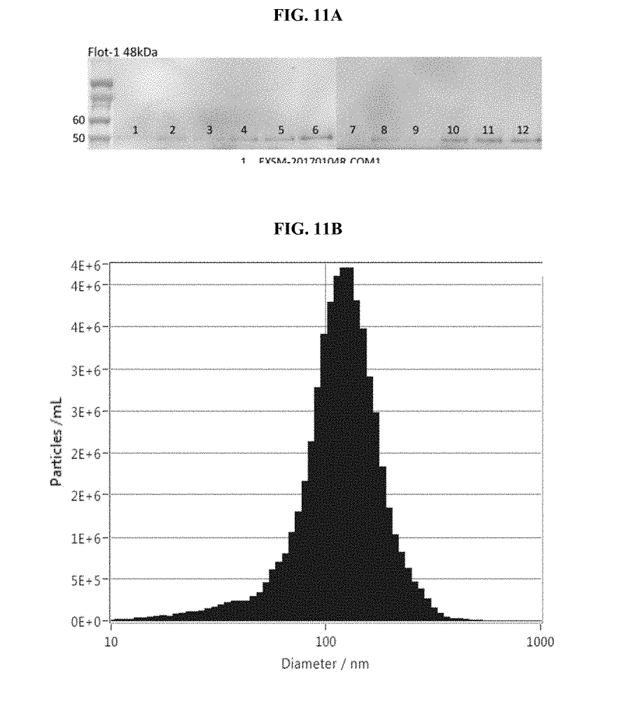

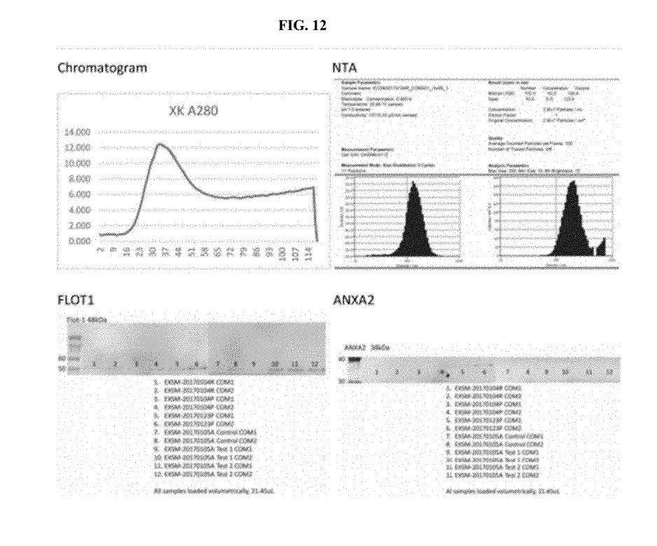

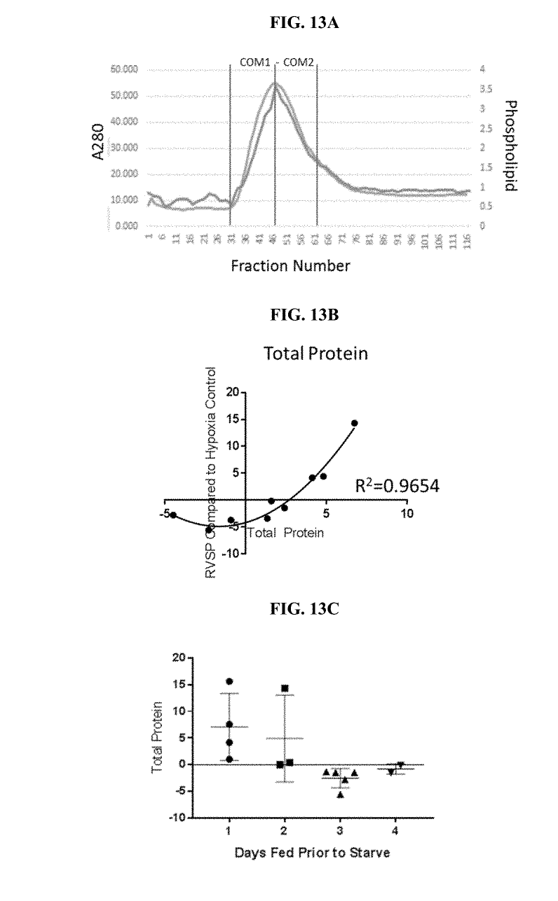

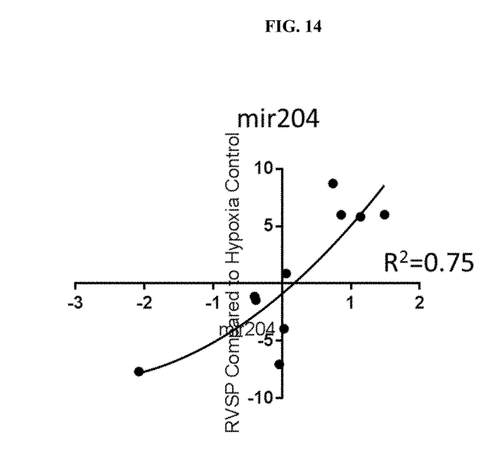

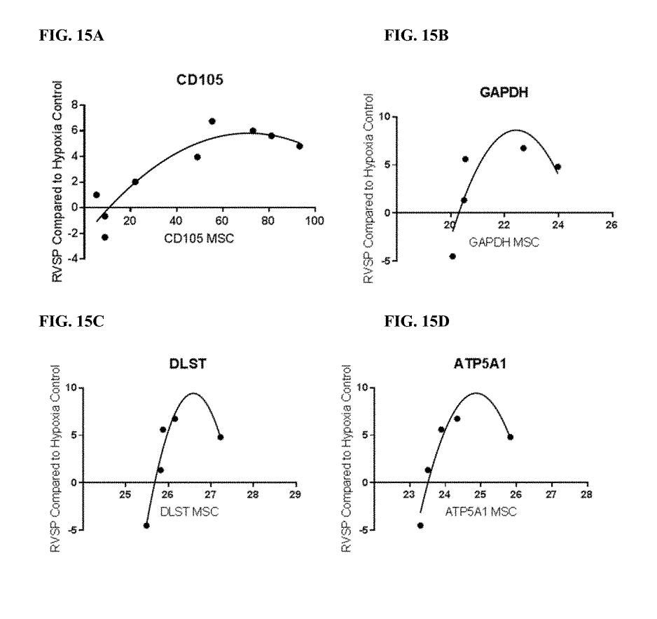

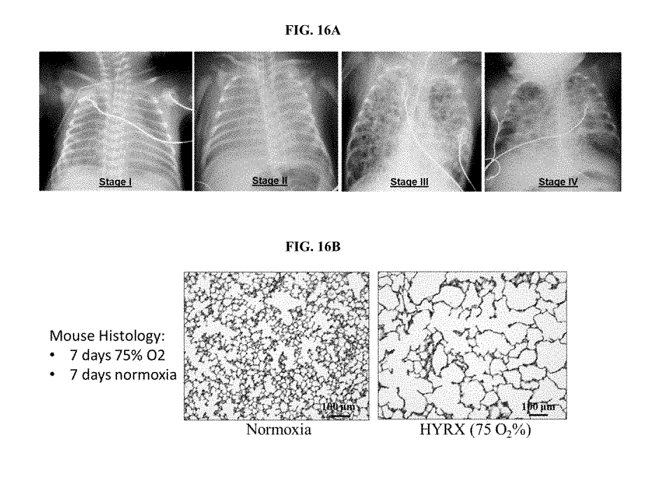

Some MSC extracellular vesicles or exosomes (e.g. bone marrow MSC extracellular vesicles or exosomes) can enhance glucose oxidation and normalize mitochondrial function. Thus, these extracellular vesicles or exosomes can confer therapeutic benefit in PAH and diseases or conditions associated with mitochondrial dysfunction. The present inventors isolated potent extracellular vesicle or exosome populations, which effectively prevented hypoxia-induced PAH in mice. Proteomics and RNAseq analysis of the potent extracellular vesicle or exosome populations show that they contain higher expression levels of genes in the glycolysis pathway, the TCA cycle and the electron transport chain. In particular, the potent extracellular vesicles or exosomes have increased expression levels of pyruvate kinase (PKM2) and ATPase, as well as their corresponding enzymatic activities. The present inventors also discovered that exposure of pulmonary artery smooth muscle cells (SMC) to acute hypoxia leads to the up-regulation of multiple genes involved in glycolysis, the TCA cycle, and the electron transport chain. Treatment of SMCs with the potent population of extracellular vesicles or exosomes prior to the hypoxia challenge normalized these genetic signatures. Furthermore, based on global metabolomics analysis, the potent population of extracellular vesicles or exosomes enhances glycolysis and ATP production in hypoxia-exposed SMCs. Without wishing to be bound by the theory, the potent population of extracellular vesicles or exosomes may improve mitochondrial function in target cells through both genetic reprograming and protein integration within key pathways, such as the glycolysis pathway, the TCA cycle, and/or the electron transport chain (see FIG. 9).

In some embodiments, the extracellular vesicles or exosomes of the present invention increase the expression of PDH and GLUD1, and therefore increase flux into the TCA cycle. Without wishing to be bound by the theory, the potent population of extracellular vesicles or exosomes may increase the expression of PDH and GLUD1 by inhibition of SIRT4, which is a known inhibitor of both PDH and GLUD1. Thus, in some embodiments, the extracellular vesicles or exosomes increase TCA cycle function.

It is contemplated that the present invention can be applied in treating pulmonary hypertension, including PAH, as well as treatment of diseases and conditions associated with mitochondrial dysfunction.

A. Definition

Unless otherwise specified, “a” or “an” means “one or more.”

Unless specifically defined otherwise, all technical and scientific terms used herein shall be taken to have the same meaning as commonly understood by one of ordinary skill in the art.

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

As used herein, the term “subject” (also referred to herein as a “patient”) includes warm-blooded animals, preferably mammals, including humans. In a preferred embodiment, the subject is a primate. In an even more preferred embodiment, the subject is a human.

As used herein the terms “treating”, “treat,” or “treatment” include reducing, mitigating, or eliminating at least one symptom of vasculopathy.

As used herein the terms “preventing”, “prevent” or “prevention” include stopping or hinder the appearance or existence of at least one symptom of vasculopathy.

As used here, the term “expression” means RNA expression and/or protein expression level of one or more genes. In other words, the term “expression” can refer to either RNA expression or protein expression.

As used here, the term “hypoxia” refers to a condition with an oxygen (O₂) concentration below atmospheric O₂ concentration, 20%. In some embodiments, hypoxia refers to a condition with O₂ concentration that is between 0% and 10%, between 0% and 5% 0₂, between 5% and 10%, or between 5% and 15%. In one embodiment, hypoxia refers to a concentration of oxygen of about 10% O₂.

As used here, the term “normoxia” refers a condition with a normal atmospheric concentration of oxygen, around 20% to 21% O₂.

As used here, the terms “isolating” or “isolated,” when used in the context of an extracellular vesicle or exosome isolated from a cell culture or media, refers to an extracellular vesicle or exosome that, by the hand of man, exists apart from its native environment.

As used here, the term “extracellular vesicles” encompasses exosomes.

As used here, the term “population of extracellular vesicles or exosomes” refers to a population of extracellular vesicles or exosomes having a distinct characteristic. The terms “population of extracellular vesicles or exosomes” and “extracellular vesicles or exosomes” can be used interchangeably to refer to a population of extracellular vesicles or exosomes having a distinct characteristic.

As used here, the term “mesenchymal stromal cell” includes mesenchymal stem cells. Mesenchymal stem cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. Mesenchymal stem cells are capable of differentiating into different germ lines such as mesoderm, endoderm, and ectoderm. Thus, mesenchymal stem cells are capable of differentiating into a large number of cell types including, but not limited to, adipose, osseous, cartilaginous, elastic, muscular, and fibrous connective tissues. The specific lineage-commitment and differentiation pathway entered into by mesenchymal stem cells depends upon various influences, including mechanical influences and/or endogenous bioactive factors, such as growth factors, cytokines, and/or local microenvironmental conditions established by host tissues. Mesenchymal stem cells are thus non-hematopoietic progenitor cells that divide to yield daughter cells that are either stem cells or are precursor cells which in time will irreversibly differentiate to yield a phenotypic cell.

Some embodiments of the invention relate broadly to mesenchymal stromal cell extracellular vesicles or exosomes, which are interchangeably referred to as mesenchymal stromal cell extracellular vesicles or exosomes, or MSC extracellular vesicles or exosomes, or extracellular vesicles or exosomes.

B. Vasculopathy

Vasculopathy includes, but is not limited to, pulmonary hypertension such as pulmonary arterial hypertension (PAH), peripheral vascular disease (PVD), critical limb ischemia (CLI), coronary artery disease, and diabetic vasculopathy.

Pulmonary hypertension, e.g. pulmonary arterial hypertension (PAH), refers to a condition in which the pressure in the lung circulation increases, eventually causing heart failure and death. Although many causes and conditions are found to be associated with PAH, many of them share in common several fundamental pathophysiological features. One feature among these processes is dysfunction of the endothelium, the internal cellular layer of all vessel walls, which is normally responsible for the production and metabolism of a large array of substances that regulate vessel tone and repair and inhibit clot formation. In the setting of PAH, endothelial dysfunction can lead to excessive production of deleterious substances and impaired production of protective substances. Whether this is the primary event in the development of PAH or part of a downstream cascade remains unknown, but in either case, it is a factor in the progressive vasoconstriction and vascular proliferation that characterize the disease. The present invention provides a method for treating pulmonary hypertension, including PAH, using isolated extracellular vesicles or exosomes.

The term peripheral vascular disease (PVD) refers to damage, dysfunction or obstruction within peripheral arteries and veins. Peripheral artery disease is the most common form of PVD. Peripheral vascular disease is the most common disease of the arteries and is a very common condition in the United States. It occurs mostly in people older than 50 years. Peripheral vascular disease is a leading cause of disability among people older than 50 years, as well as in those people with diabetes. About 10 million people in the United States have peripheral vascular disease, which translates to about 5% of people older than 50 years. The number of people with the condition is expected to grow as the population ages. Men are slightly more likely than women to have peripheral vascular disease.

Critical limb ischemia (CLI), due to advanced peripheral arterial occlusion, is characterized by reduced blood flow and oxygen delivery at rest, resulting in muscle pain at rest and non-healing skin ulcers or gangrene (Rissanen et al., Eur. J. Clin. Invest. 31:651-666 (2001); Dormandy and Rutherford, J. Vasc. Surg. 31:S1-S296 (2000)). Critical limb ischemia is estimated to develop in 500 to 1000 per million individuals in one year (“Second European Consensus Document on Chronic Critical Leg Ischemia”, Circulation 84(4 Suppl.) IV 1-26 (1991)). In patients with critical limb ischemia, amputation, despite its associated morbidity, mortality and functional implications, is often recommended as a solution against disabling symptoms (M. R. Tyrrell et al., Br. J. Surg. 80: 177-180 (1993); M. Eneroth et al., Int. Orthop. 16: 383-387 (1992)). There exists no optimal medical therapy for critical limb ischemia (Circulation 84(4 Suppl.): IV 1-26 (1991)).

Coronary artery disease (atherosclerosis) is a progressive disease in humans wherein one or more coronary arteries gradually become occluded through the buildup of plaque. The coronary arteries of patients having this disease are often treated by balloon angioplasty or the insertion of stents to prop open the partially occluded arteries. Ultimately, these patients are required to undergo coronary artery bypass surgery at great expense and risk.

Bronchopulmonary Dysplasia (BPD) is a chronic lung disease of premature infants. It is characterized by prolonged lung inflammation, decrease in number of alveoli and thickened alveolar septae, abnormal vascular growth with “pruning” of distal blood vessels, and limited metabolic and anti-oxidant capacity. There are 14,000 new cases of BPD per year in the US. Importantly, a diagnosis of BPD often leads to other further conditions, including PAH, emphysema, asthma, increase cardiovascular morbidity and post-neonatal mortality, increased neurodevelopmental impairment and cerebral palsy, emphysema as young adults. Currently, there is no standard therapy for BPD. Some BPD patients are treated with gentle ventilation and corticosteroids, but these treatments show no effects on neuro outcomes or death. The primary risk for BPD exists in infants between 24-28 weeks after birth, which correspond to the period of the beginning of saccular development. The infants at high risk are of 1.3 to 2.2 pounds.

In one aspect, the exosomes of the present invention may be used to treat BPD. In some embodiments, the exosomes increase immunomodulatory capacity of the lung. In some embodiments, the exosomes promote angiogenesis in the lung. In some embodiments, the exosomes increase mitochondrial metabolism of the lung.

C. Mitochondrial Dysfunction

Mitochondria are intracellular organelles responsible for a number of metabolic transformations and regulatory functions. They produce much of the ATP employed by eukaryotic cells. They are also the major source of free radicals and reactive oxygen species that cause oxidative stress. Consequently, mitochondrial defects are damaging, particularly to neural and muscle tissues, which have high energy level demands. Thus, energetic defects have been implicated in forms of movement disorders, cardiomyopathy, myopathy, blindness, and deafness (DiMauro et al. (2001) Am. J. Med. Genet.106, 18-26; Leonard et al. (2000) Lancet.355, 299-304). Mitochondrial dysfunction can involve increased lactate production, diminished respiration and ATP production. Mitochondrial dysfunction can manifest in consequences of oxidative stress.

The present invention provides methods for treating diseases or conditions associated with mitochondrial dysfunction. Mitochondrial dysfunction can be associated with decreased mitochondrial glucose oxidation in the subject.

In some embodiments, the disease or condition associated with mitochondrial dysfunction is selected from the group consisting of Friedreich's ataxia, Leber's Hereditary Optic Neuropathy, Kearns-Sayre Syndrome, Mitochondrial Encephalomyopathy with Lactic Acidosis and Stroke-Like Episodes, Leigh syndrome, obesity, atherosclerosis, amyotrophic lateral sclerosis, Parkinson's Disease, cancer, heart failure, myocardial infarction (MI), Alzheimer's Disease, Huntington's Disease, schizophrenia, bipolar disorder, fragile X syndrome, and chronic fatigue syndrome.

D. Mitochondrial Energy Production

Cells in eukaryotic organisms require energy to carry out cellular processes. Such energy is mainly stored in the phosphate bonds of adenosine 5 '-triphosphate (“ATP”). There are certain pathways that generate energy in eukaryotic organisms, including: (1) glycolysis; (2) the TCA cycle (also referred to as Krebs Cycle or citric acid cycle); and (3) oxidative phosphorylation. For ATP to be synthesized, carbohydrates are first hydrolyzed into monosaccharides (e.g., glucose), and lipids are hydrolyzed into fatty acids and glycerol. Likewise, proteins are hydrolyzed into amino acids. The energy in the chemical bonds of these hydrolyzed molecules are then released and harnessed by the cell to form ATP molecules through numerous catabolic pathways.

The main source of energy for living organisms is glucose. In breaking down glucose, the energy in the glucose molecule's chemical bonds is released and can be harnessed by the cell to form ATP molecules. The process by which this occurs consists of several stages. The first is called glycolysis, in which the glucose molecule is broken down into two smaller molecules called pyruvic acid.

In glycolysis, glucose and glycerol are metabolized to pyruvate via the glycolytic pathway. During this process, two ATP molecules are generated. Two molecules of NADH are also produced, which can be further oxidized via the electron transport chain and result in the generation of additional ATP molecules.

Glycolysis involves many enzyme-catalyzed steps that break down glucose (and other monosacharrides) into 2 pyruvate molecules. In return, the pathway leads to the generation of a sum of 2 ATP molecules. The pyruvate molecules generated from the glycolytic pathway enter the mitochondria from the cytosol. The molecules are then converted to acetyl co-enzyme A (Acetyl-CoA) for entry into the TCA cycle. The TCA cycle consists of the bonding of acetyl coenzyme-A with oxaloacetate to form citrate. The formed citrate is then broken down through a series of enzyme-catalyzed steps to generate additional ATP molecules.

Energy released from the TCA cycle in the mitochondrial matrix enters the mitochondrial electron transport chain as NADH (complex I) and FADH₂ (complex II). These are the first two of five protein complexes involved in ATP production, all of which are located in the inner mitochondrial membrane. Electrons derived from NADH (by oxidation with a NADH-specific dehydrogenase) and FAD¾ (by oxidation with succinate dehydrogenase) travel down the respiratory chain, releasing their energy in discrete steps by driving the active transport of protons from the mitochondrial matrix to the intermembrane space (i.e., through the inner mitochondrial membrane). The electron carriers in the respiratory chain include flavins, protein-bound iron-sulfur centers, quinones, cytochromes and copper. There are two molecules that transfer electrons between complexes: coenzyme Q (complex I→III, and complex II→III) and cytochrome c (complex III→IV). The final electron acceptor in the respiratory chain is (¾, which is converted to ¾0 in complex IV.

Some embodiments of the present invention relate to extracellular vesicles or exosomes that have increased expression of at least one genes or proteins in glycolysis, the TCA cycle, and/or the electron transport chain. In some embodiments, the genes are selected from the group of genes represented by Table 1 below.

TABLE 1

| Pathway | Proteins | Genes |
|---|---|---|
| Glycolysis | AGI, ALDO, TPI, | ALDOA, ENO3, GPI, |
| | GAPDH, ENO, | HK2,3, PFK, PGM, PK |
| | PGAM, PK | |
| TCA Cycle | OGDH | MDH2, OGDH, PC, PDHA1, |
| | | PDHB, SDHA, SDHC, SUCLG2 |
| Electron | ETFA, ATPase | Complex I (NDUFC2, NDUFB1, |
| Transport | | NDUFS5, NDUFA8, NDUFA9, |
| Chain | | NDUFS2); Complex II (SDHA, |
| | | SDHC); Complex III (UQCRH1); |
| | | Complex IV (Cox 6c1, Cox10); |
| | | Complex V (ATPase genes) |

In some embodiments, the gene in the glycolysis pathway is selected from the group consisting of PK, AGI, ALDO, ALDOA, ENO3, GPI, HK2, HK3, PFK, PGM, TPI, GAPDH, ENO, and PGAM.

In some embodiments, the gene in the TCA cycle is selected from the group consisting of MDH2, OGDH, PC, PDHA1, PDHB, SDHA, SDHC, and SUCLG2.

In some embodiments, the gene in the electron transport chain is selected from the group consisting of ETFA, ATPase, NDUFC2, NDUFB1,NDUFS5, NDUFA8, NDUFA9, NDUFS2, SDHA, SDHC, UQCRH1, Cox 6c1, and Cox10.

In some embodiments, the extracellular vesicles or exosomes have increased expression of PK.

In some embodiments, the extracellular vesicles or exosomes have increased expression of ATPase.

In some embodiments, the extracellular vesicles or exosomes have increased expression of PK and ATPase.

E. Extracellular Vesicles Isolated from Mesenchymal Stromal Cells

The extracellular vesicles or exosomes of the invention can be, for example, membrane (e.g., lipid bilayer) vesicles that are released from mesenchymal stromal cells. They can have, for example, a diameter ranging from about 30 nm to 100 nm. By electron microscopy, extracellular vesicles or exosomes can appear to have a cup-shaped morphology. They can, for example, sediment at about 100,000×g and have a buoyant density in sucrose of about 1.10 to about 1.21 g/ml.

Mesenchymal stromal cells may be harvested from a number of sources including but not limited to bone marrow, blood, periosteum, dermis, umbilical cord blood and/or matrix (e.g., Wharton's Jelly), and placenta. Methods for harvest of mesenchymal stromal cells are described in greater detail in the Examples. Reference can also be made to U.S. Pat. No. 5,486,359, which is incorporated herein by reference, for other harvest methods that can be used in the present invention.

The mesenchymal stromal cells, and thus the extracellular vesicles or exosomes, contemplated for use in the methods of the invention may be obtained from the same subject to be treated (and therefore would be referred to as autologous to the subject), or they may be obtained from a different subject, preferably a subject of the same species (and therefore would be referred to as allogeneic to the subject).

As used herein, it is to be understood that aspects and embodiments of the invention relate to cells as well as cell populations, unless otherwise indicated. Thus, where a cell is recited, it is to be understood that a cell population is also contemplated unless otherwise indicated.

Some aspects of the invention refer to isolated extracellular vesicles or exosomes. As used herein, an isolated extracellular vesicle or exosome is one which is physically separated from its natural environment. An isolated extracellular vesicle or exosome may be physically separated, in whole or in part, from a tissue or cellular environment in which it naturally exists, including mesenchymal stromal cells. In some embodiments of the invention, a composition of isolated extracellular vesicles or exosomes may be free of cells such as mesenchymal stromal cells, or it may be free or substantially free of conditioned media. In some embodiments, the isolated extracellular vesicles or exosomes may be provided at a higher concentration than extracellular vesicles or exosomes present in un-manipulated conditioned media. Extracellular vesicles or exosomes may be isolated from conditioned media from mesenchymal stromal cell culture.

Generally any suitable method for purifying and/or enriching extracellular vesicles or exosomes can be used, such as methods comprising magnetic particles, filtration, dialysis, ultracentrifugation, ExoQuick™ (Systems Biosciences, Calif., USA), and/or chromatography. In some embodiments, extracellular vesicles or exosomes are isolated by centrifugation and/or ultracentrifugation. Extracellular vesicles or exosomes can also be purified by ultracentrifugation of clarified conditioned media. They can also be purified by ultracentrifugation into a sucrose cushion. The protocol is described in, for example, Thery et al. Current Protocols in Cell Biol. (2006) 3.22, which is incorporated herein by reference. In some embodiments, extracellular vesicles or exosomes are isolated by a single step size exclusion chromotography. The protocol is described in, for example, Boing et al. Journal of Extracellular Vesicles (2014) 3:23430, which is incorporated herein by reference. A detailed method for harvest of extracellular vesicles or exosomes from mesenchymal stromal cells or mesenchymal stem cells is provided in the Examples.

The invention also contemplates immediate use of extracellular vesicles or exosomes or alternatively short- and/or long-term storage of extracellular vesicles or exosomes, for example, in a cryopreserved state prior to use. Proteinase inhibitors are typically included in freezing media as they provide extracellular vesicle or exosome integrity during long-term storage. Freezing at −20° C. is not preferable since it is associated with increased loss of extracellular vesicle or exosome activity. Quick freezing at −80° C. is more preferred as it preserves activity. See for example Kidney International (2006) 69, 1471-1476, which is incorporated herein by reference. Additives to the freezing media may be used in order to enhance preservation of extracellular vesicle or exosome biological activity. Such additives will be similar to the ones used for cryopreservation of intact cells and may include, but are not limited to DMSO, glycerol and polyethylene glycol.

F. Assessment of the Potency of Extracellular Vesicles or Exosomes

The present invention provides using right ventricular systolic pressure (RVSP) to measure the effect of extracellular vesicle or exosome treatment on hypoxia induced PAH mice model, and to identify potent populations of extracellular vesicles or exosomes. In some embodiments, the potent populations of extracellular vesicles or exosomes are capable of reducing RVSP of mice subjected to a three-week chronic hypoxia exposure by at least about 10%, 12.5%, 15%, 17.5%, 20%, 22.5%, 25%, 27.5%, or 30%, compared to control mice subjected to a three-week chronic hypoxia exposure and treated with PBS buffer.

In some embodiments, the potent populations of extracellular vesicles or exosomes are identified by delta RVSP. As used here, delta RVSP is defined as the RVSP of hypoxia-exposed mice treated with extracellular vesicles or exosomes minus RVSP of normoxia mice. In some embodiments, a population of extracellular vesicles or exosomes is potent if delta RVSP is less than about 6, 5, 4, 3, or 2 mmHg.

In some embodiments, the potency of populations of extracellular vesicles or exosomes are characterized by their ability to increase O₂ consumption by smooth muscle cells (SMC) lysates. In some embodiments, the potent populations of extracellular vesicles or exosomes are capable of increasing O₂ consumption by SMC lysate subjected to a 24-hour hypoxia exposure by at least about 10%, 15%, 20%, 25%, 30%, 35%, or 40%, compared to control SMC cell lysates subjected to a 24-hour hypoxia exposure and treated with PBS control.

In some embodiments, the potency of populations of extracellular vesicles or exosomes is characterized by their PK activity. In some embodiments, the potent population of extracellular vesicles or exosomes have a PK activity of at least about 0.15 nmol/min/ml, 0.16 nmol/min/ml, 0.17 nmol/min/ml, 0.18 nmol/min/ml, 0.19 nmol/min/ml, 0.20 nmol/min/ml, 0.21 nmol/min/ml, 0.22 nmol/min/ml, 0.23 nmol/min/ml, 0.24 nmol/min/ml, 0.25 nmol/min/ml, 0.3 nmol/min/ml, or 0.4 nmol/min/ml.

In some embodiments, the potency of populations of extracellular vesicles or exosome are characterized by their LDH activity. In some embodiments, the potency of populations of extracellular vesicles or exosome are characterized by their ability to decrease LDH secreted by hypoxia-exposed SMC by at least about 10%, 20%, 30%, or 40%.

In some embodiments, the extracellular vesicles or exosome of the present invention are isolated based on one or more criteria in the table below:

| | |
|---|---|
| Sterility | PT-PCR mir204 |
| Appearance | MSC-CD105 |
| Total Protein | PT-PCR mitochondrial genes |
| Particle Count | 2D gel electrophoresis |
| Total RNA | RNAseq fingerprint analysis |
| Total Phospholipid | In vitro potency assay |

In some embodiments, the isolated extracellular vesicles or exosomes comprise an amount of mir204 that is at least 10%, 20%, 30%, 50%, or 100% more than the average level of mir204 in all extracellular vesicles or exosomes of the mesenchymal stromal cells.

In some embodiments, the isolated extracellular vesicles or exosomes comprise an amount of CD105, GAPDH, DLST, and/or ATP5Althat is at least 10%, 20%, 30%, 50%, or 100% more than the average level of CD105, GAPDH, DLST, and/or ATP5A1 in all extracellular vesicles or exosomes of the mesenchymal stromal cells.

In some embodiments, the isolated extracellular vesicles or exosomes comprise an amount of RNA expression of SORCS1, FHIT and/or ANKRD30 BL that is at least 10%, 20%, 30%, 50%, or 100% more than the average level of RNA expression of SORCS1, FHIT and/or ANKRD30 BL in all extracellular vesicles or exosomes of the mesenchymal stromal cells.

In some embodiments, the isolated extracellular vesicles or exosomes have reduced MHCII contaminants or are substantially or totally free of MHCII contaminants, such as comprising an amount of MHCII contaminants that is at least 50%, 70%, 80%, 90%, 95%, 98%, or 99% less than the average level of MHCII contaminants in all extracellular vesicles or exosomes of the mesenchymal stromal cells.

In some embodiments, the isolated extracellular vesicles or exosomes have reduced fibronectin content or are substantially or totally free of fibronectin, such as comprising an amount of fibronectin that is at least 50%, 70%, 80%, 90%, 95%, 98%, or 99% less than the average level of fibronectin in all extracellular vesicles or exosomes of the mesenchymal stromal cells.

G. Treatment Using Extracellular Vesicles or Exosomes

Compositions useful for the methods of the present disclosure can be administered via, inter alia, localized injection, including catheter administration, systemic injection, localized injection, intravenous injection, intrauterine injection or parenteral administration. When administering a therapeutic composition described herein (e.g., a pharmaceutical composition), it will generally be formulated in a unit dosage injectable form (e.g. solution, suspension, or emulsion).

The invention contemplates single or repeated administration of extracellular vesicles or exosomes, including two, three, four, five or more administrations of extracellular vesicles or exosomes. In some embodiments, the extracellular vesicles or exosomes may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ. As an example, if the symptoms of the disease appear to be worsening the extracellular vesicles or exosomes may be administered more frequently, and then once the symptoms are stabilized or diminishing the extracellular vesicles or exosomes may be administered less frequently.

The invention also contemplates repeated administration of low dosage forms of extracellular vesicles or exosomes as well as single administrations of high dosage forms of extracellular vesicles or exosomes. Low dosage forms may range from, without limitation, 1-50 micrograms per kilogram, while high dosage forms may range from, without limitation, 51-1000 micrograms per kilogram. It will be understood that, depending on the severity of the disease, the health of the subject, and the route of administration, inter alia, the single or repeated administration of low or high dose extracellular vesicles or exosomes are contemplated by the invention.

The extracellular vesicles or exosomes may be used (e.g., administered) in pharmaceutically acceptable preparations (or pharmaceutically acceptable compositions), typically when combined with a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, or solvent encapsulating material.

Such preparations may routinely contain pharmaceutically acceptable concentrations of salt, buffering agents, preservatives, compatible carriers, and may optionally comprise other (i.e., secondary) therapeutic agents. A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations.

The preparations of the invention are administered in effective amounts. An effective amount is that amount of an agent that alone stimulates the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The invention also encompasses a packaged and labelled pharmaceutical product. This article of manufacture or kit includes the appropriate unit dosage form in an appropriate vessel or container such as a glass vial or plastic ampoule or other container that is hermetically sealed. The unit dosage form should be suitable for pulmonary delivery for example by aerosol. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other monitoring information.

As with any pharmaceutical product, the packaging material and container are designed to protect the stability of the product during storage and shipment. The kits may include MSC extracellular vesicles or exosomes in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant.

### EXAMPLES

The following examples are intended to provide those of ordinary skill in the art with a complete disclosure and description of how to make and use the methods and compositions described herein, and are not intended to be limiting.

### Example 1

### Isolating Exosomes Populations

This example demonstrates isolation of exosomes from a cell culture media.

Filtration: Conditioned media obtained from mesenchymal stem cells (MSCs) was collected in a 5 L Sartorius-stedium Flexboy® bag with 0.2 um filters. The collected conditioned media was pumped through a filter line to quickly eliminate any cells, dead cells, and cellular debris. The, the condition media was supplemented with 25 mM HEPES and 10 mM EDTA buffers using a 140 ml luer-lok syringe.

Tangential flow filtration: The 5 L Flexboy® bag containing the conditioned media was connected to a tangential flow filtration (TFF) system, by a sample line attached to the Flexboy bag and connected to the top of the TFF reservoir. A Sartorius Sartocon Slice TFF with a single 100 kDa MWCO 0.1m²Hydrosart® cassette was used. A water integrity test was conducted at the beginning and at the end of each TFF run to measure the integrity of the cassette. The system was then primed with 1 L of PBS. The media sample was then gravity fed into the reservoir. The TFF was run at 600 LMH. An initial media of 5 L volume was concentrated down to 100 mL (a 50× concentration). The retentate was collected and filtered using a 0.22 um filter. The filtrate was divided into 10 mL aliquot sample and frozen at −80° C.

Fractionation: Samples were thawed at 37° C. for approximately 10 minutes. All samples were pooled together in a 150 mL corning bottle. A XK 50/100 column was packed using Sepharose CL-2B resin (GE). The XK 50/100 column was connected to an AKTA Aant 150 (GE). The sample was introduced into the column via the sample line. Once all the sample was introduced to the column, the elution step began (settings: flow rate of 4.6 ml/min). 0.2CV of void column eluted out and then the fraction collector started collecting fractions at a rate of 1 minute per fraction (4.6 mL in each fraction). Fractions were collected until 0.6CV was eluted out (exosomes eluted out between 0.3CV-0.4CV). PBS was used for the entire experiment. The fraction samples are capped under the hood and stored at 4° C.

Diafiltration: Samples may be optionally subjected to a diafiltration step, preferably after the TFF step and before the Fractionation step which is similar to buffer exchange. Once a desired concentration of exosomes is reached, PBS buffer was added to the sample through a reservoir to maintain the volume while continuing to run the pump to the TFF cassette filter. Gradually, the PBS replaced the conditioned media. In order to achieve as complete of an exchange as possible, 7 total volume diafiltrations were performed to with the retentate. This step helps to remove some of the impurities in the retentate, without affecting exosome. The presence of exosomes was verified by FLOT-1 western blots, which shows decreased amount of total protein and phospholipid.

Measuring phospholipid concentration: Phospholipid signaling was used for exosome detection. Briefly, after fractionation, 20 uL of each exosome prep and 80 uL of a reaction mix (Sigma) were transferred into black, clear-bottom 96-well plates (Corning, Corning, N.Y.) and incubated for 30 minutes at room temperature protected from light. Fluorescence intensity was measured at 530/585 nm using a FLUOstar Omega microplate reader (BMG Labtech, Ortenberg, Germany). In the exosome production runs shown, both A280 chromatograms and phospholipid were utilized for exosome detection.

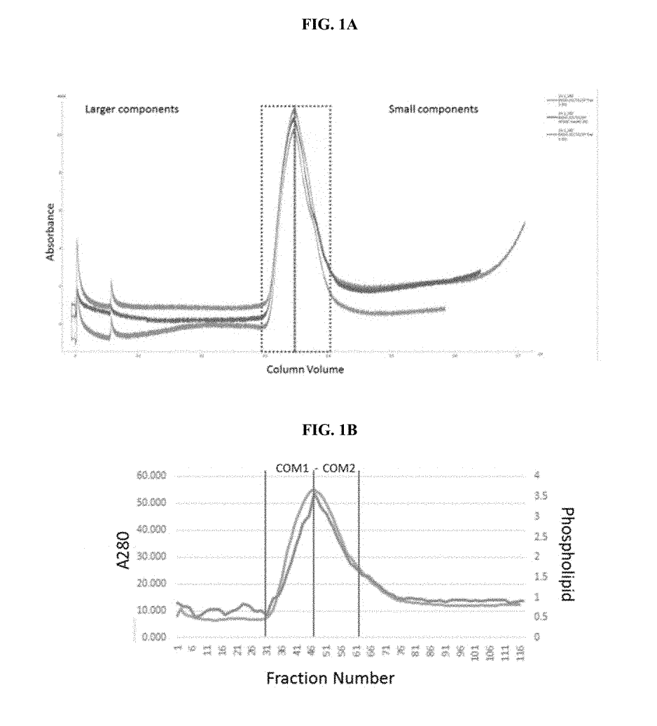

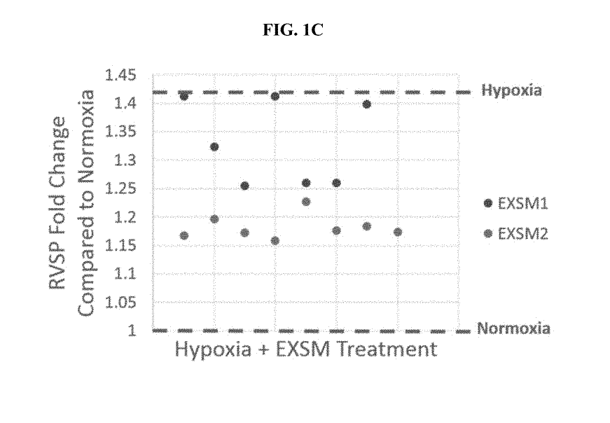

As shown in FIG. 1C, phospholipid signaling overlays well with A280 chromatograms for exosome detection.

### Example 2

### Treating Pulmonary Arterial Hypertension (PAH) in Mouse Model

Mice were subjected to a three week chronic hypoxia exposure to induce PAH (shown as an increase in Right Ventricular Systolic Pressure). Exosomes treatment consisted of a 1 time tail vein injection prior to hypoxia exposure.

### Example 3

### Identification of Potent Exosome Populations

To identify potent exosome populations, each exosome preparation was analyzed for pyruvate kinase protein expression using a PKM2 antibody (Cell Signaling, Danvers, Mass.). A capillary electrophoresis immunoassay was performed using the WESTM machine (ProteinSimple, San Jose, Calif.) according to the manufacturer's protocol. In brief, 4.2 uL of samples were mixed 1:5 with a master fluorescent mix (ProteinSimple). Samples were then heated at 95° C. for 5 min and placed on ice. The primary PKM2 antibody was diluted 1:15 in antibody diluent (ProteinSimple) and a proprietary anti-Rabbit Secondary antibody (ProteinSimple) was used. Proprietary peroxide and luminol-S (ProteinSimple) were mixed 1:1 to make the chemiluminescent substrate. The samples, blocking reagent, primary antibody, secondary antibody, chemiluminescent substrate, and wash buffer were loaded into designated wells in the provided microplate. The plate was spun at 1,000 g for 5 minutes to avoid bubbles in wells. The plate and capillary cartridges were loaded into the WES machine. After plate loading, fully automated electrophoresis and immunodetection took place in the capillary system. Proteins were separated using WES standard run settings. The data was analyzed with built-in Compass software (Proteinsimple), providing peak molecular weight signal and area under the curve values per sample.

Pyruvate Kinase Activity: Pyruvate kinase is an enzyme in glycolysis which catalyzes the transfer of phosphate from phosphoenolpyruvate (PEP) to ADP, yielding one molecule of pyruvate and one molecule of ATP. Pyruvate kinase is measured by the abcam kit (ab83432) wherein PEP and ADP are catalyzed by PK to generate pyruvate and ATP. The generated pyruvate is oxidized by pyruvate oxidase to produce color (at λ=570 nm). Because color intensity is proportional to pyruvate amount, PK activity can be measured. PK activity generates is a kinetic assay. Data analysis can be done using the following equation:

PK activity=(pyruvate×dilution factor)/(T2-T1)×well volume

Where T2-T1 is time (mins) at timepoint 2-timepoint 1. Pyruvate (nmol) is calculated using a pyruvate standard curve (where pyruvate is calculated as final pyruvate concentration at T2 minus initial pyruvate concentration at T1). This number needs to be blank corrected. It is important that the activity measures occur within the linear range. The dataset can be analyzed at multiple time points. The best way to selected is to look at the curves and choose data points at least 2 minutes apart that fall within the linear range. Every sample within the plate is analyzed the same way. In this experiment, T1=2 min, T2=4 minutes were chosen, as these two time points were well within the linear range.

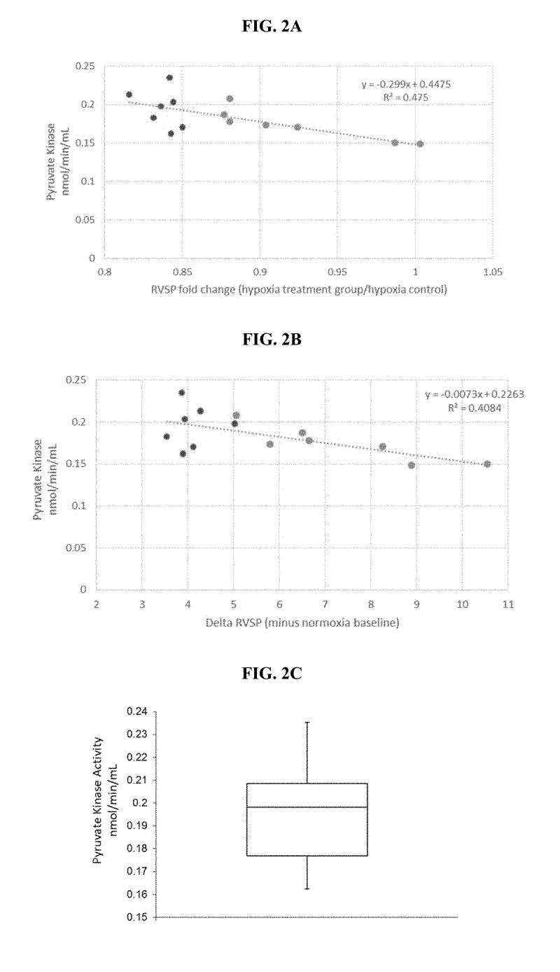

Next, pyruvate kinase activity was plotting against the in vivo RVSP fold change of each exosome preparation treatment condition over the hypoxia control. This allows for the comparison of pyruvate kinase activity to the fold improvement in RVSP with exosome treatment. See FIG. 2A. Pyruvate kinase activity was also plotted against the delta RVSP. Delta RVSP is the hypoxia treated with exosome condition minus normoxia control. See FIG. 2B. The red dots represent potent exosome populations that induced a significant improvement in RVSP. The blue dots represent exosome populations that are not potent in treating hypoxia induced PAH mice. The pyruvate kinase activity of these most potent exosome populations was then graphed in a box and whisker plot. See FIG. 2C. Therefore, the graph in (C) represents the pyruvate kinase activity range of our most potent exosome preps.

### Example 4

### Proteomic Analysis and RNAseq Analysis of Potent Exosome Populations

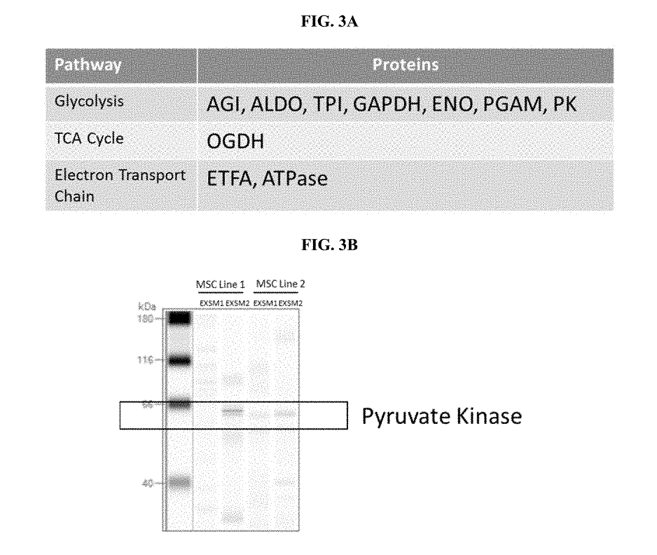

Samples exosomes were sent to Bioproximity (Chantilly, Va.) for proteomics analysis. The proteins with increased expression levels in the potent exosome population compared to the non-potent population are shown in FIG. 3(a).

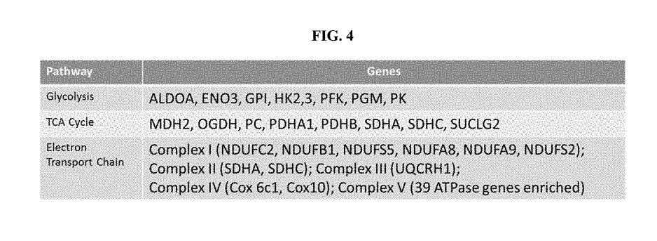

Samples exosomes were sent to SBI for RNAseq analysis. The genes with increased expression levels in the potent exosome population compared to the non-potent population are shown in FIG. 4.

### Example 5

### Measuring Extracellular O₂ Consumption

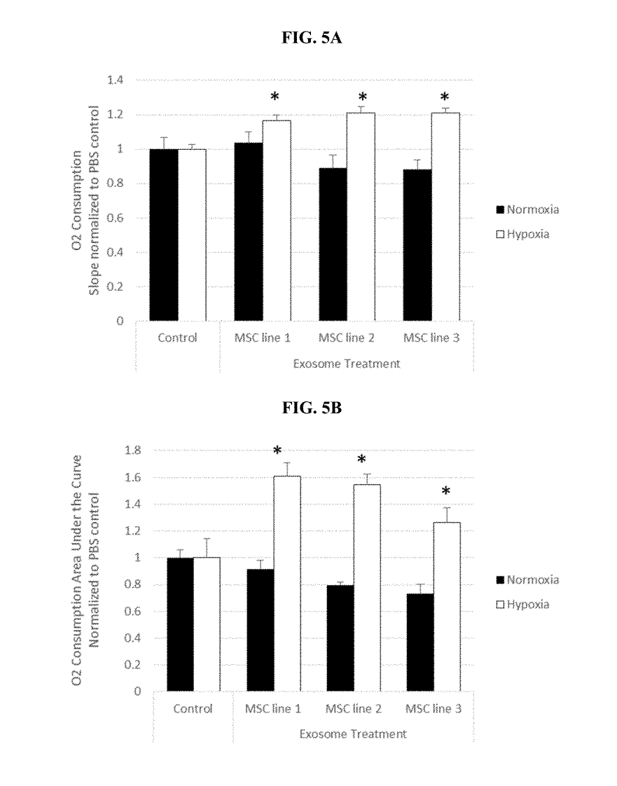

The abcam Extracellular O₂ Consumption Assay Kit (ab197243) was used to measure oxygen consumption of Smooth muscle cells (SMC) lysates treated with PBS (control) or exosome after 24 hour exposure to either normoxia or 4% O₂ hypoxia. As the cell lysates consume oxygen via the electron transport chain, oxygen was depleted in the surrounding culture media which is seen as an increase in phosphorescence signal. The micro-environment is protected from ambient air diffusion by addition of mineral oil to each well and phosphorescence signal is measured as a quenching of the O₂ probe supplied by the manufacturer. These data were calculated two ways: both as area under the curve and as the slope over time (data analysis using the slope is recommended by the manufacturer). FIGS. 5A-5B demonstrate that exosomes do not significantly change the cellular O₂ consumption in normoxia-exposed SMCs. However, exosome treatment induces an increase in O₂ consumption under hypoxia stress, which indicates an increase in mitochondrial function.

### Example 6

### Microarray Analysis of SMC Exposed to Acute Hypoxia

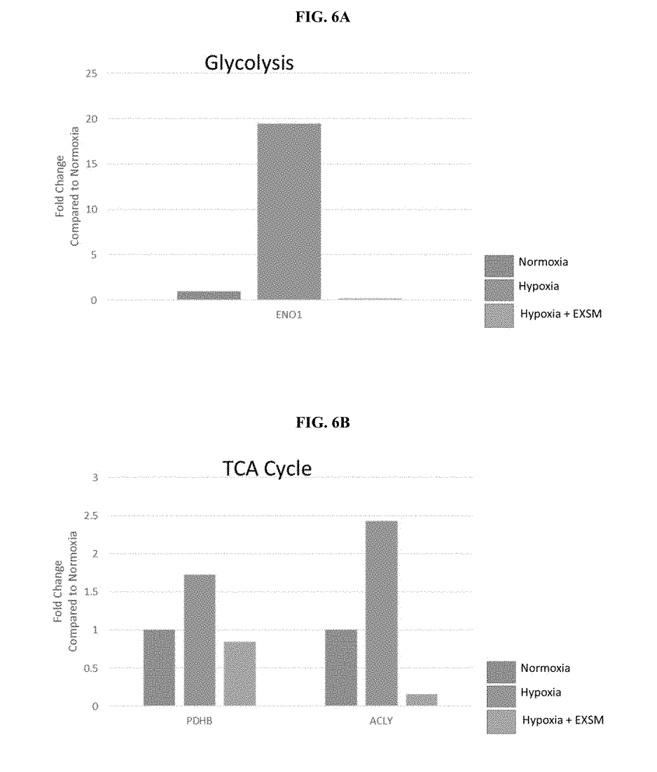

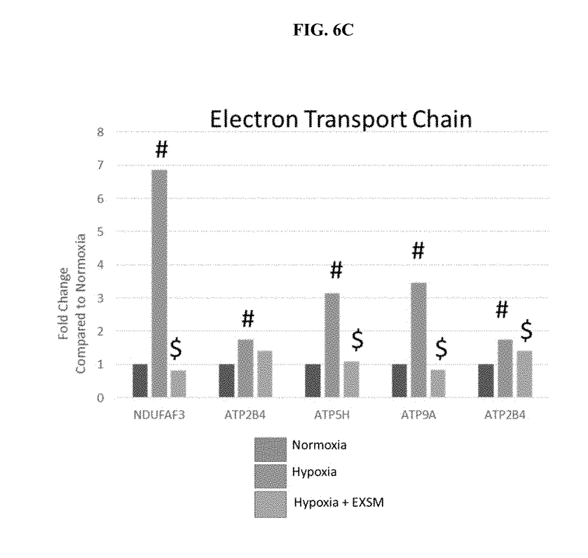

Smooth muscle cells (SMC) were treated with either PBS control or exosomes (EXSM) and incubated for 24 hours in either normoxia or hypoxia (4% O₂) conditions. RNA of SMC was isolated and sent to Qiagen for analysis using the global microarray platform. FIGS. 6A-6C shows that there is an increase in expression of genes in glucose metabolism after acute hypoxia exposure. EXSM treatment normalized this response in (a) glycolysis, (b) the TCA cycle and (c) the electron transport chain. These data suggest exosome treatment decreased or eliminated the need for genetic up-regulation of glucose oxidation genes during hypoxia stress, likely due to EXSM protein or gene incorporation into the pathways.

### Example 7

### Metabolomics Analysis of SMC Exposed to Acute Hypoxia

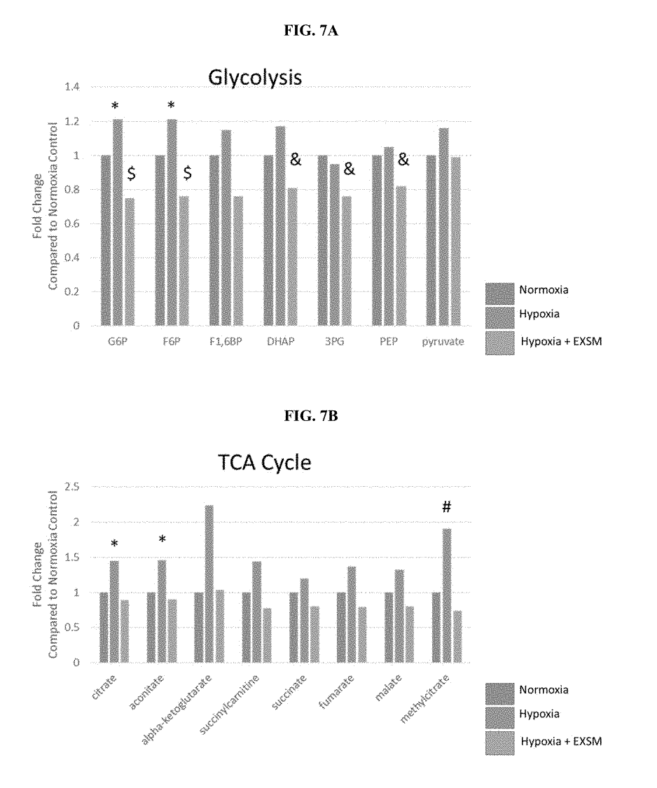

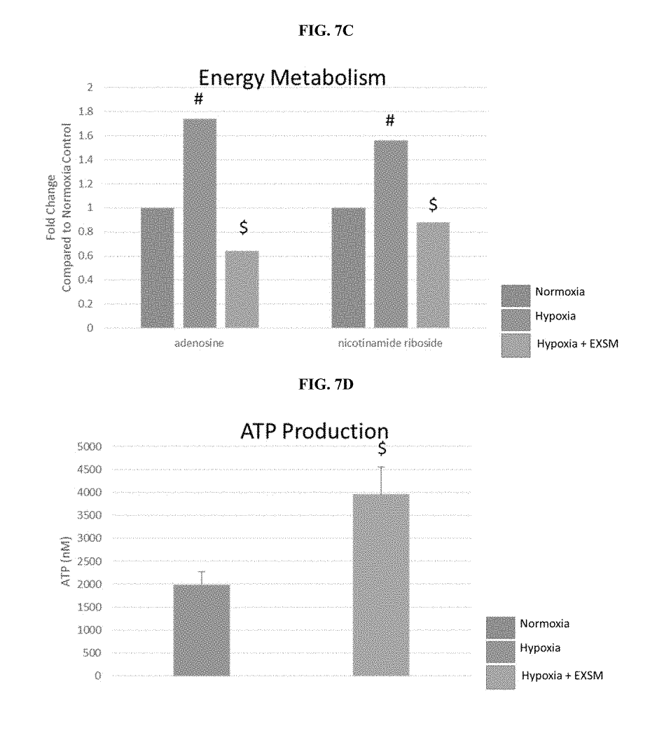

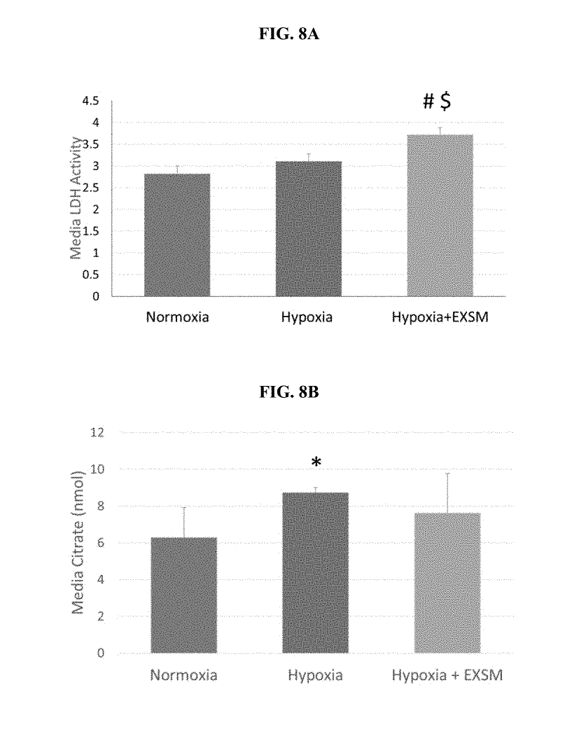

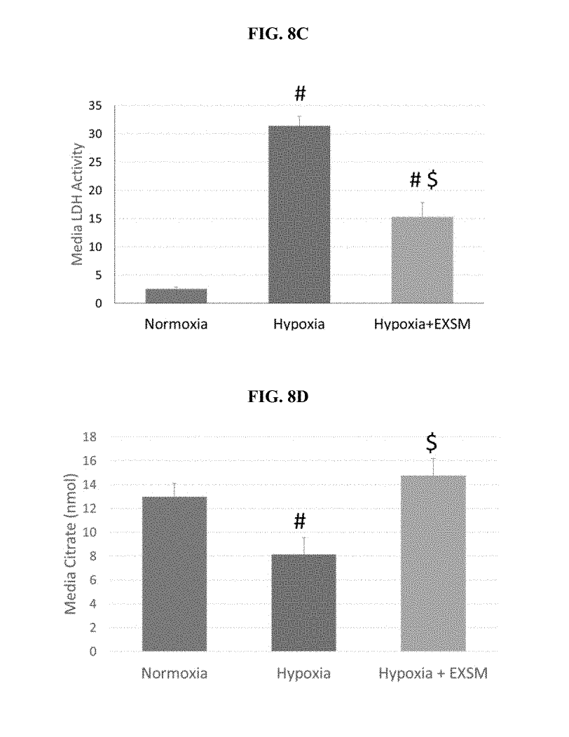

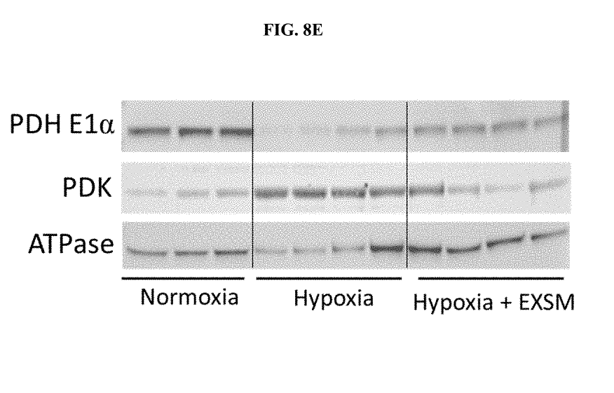

Smooth muscle cells (SMC) were treated with either PBS control or exosomes (EXSM) and incubated for 24 hours in either normoxia or hypoxia (4% O₂) conditions. Cell lysates were pelleted and sent to Metabolon (North Carolina) for global metabolite analysis. As shown in FIG. 7, SMC lysates showed a build-up of metabolites in glycolysis, TCA cycle and energy metabolism pathways after acute hypoxia exposure, due to decreased flux through these pathways. Exosome treatment increased flux through (a) glycolysis and (b) the TCA cycle (represented by decreased metabolite build-up). These data indicates an increase in glucose oxidation. FIGS. 7A-7D show that acute hypoxia exposure resulted in a build-up adenosine and nicotinamide riboside, which are building blocks of ATP and NAD. Exosome treatment resulted in adenosine and nicotinamide riboside use, likely due to increased ATP production (c). ATP production was measured on live SMCs exposed to acute hypoxia (d), and confirmed an EXSM-mediated increase in ATP generation.

### Example 8

### Additional Methods

Mouse Model: C57BL/6 mice were housed in hypoxia tents with oxygen levels controlled at 10% oxygen for three weeks to induce pulmonary hypertension. For exosomes treatment, a single-dose was injected into the tail vein 3 hours prior to hypoxia exposure.

Exosome Isolation and Analysis: Serum-free conditioned media was collected from confluent MSC cultures over a 40 hour period. Conditioned media was concentrated 50× using tangential flow filtration. Concentrate was incubated with the fluorescent lipophilic dye, DiI, and then fractionated using an XK 50/100 column packed with Sepharose CL-2B resin (GE Heathcare). Exosome-containing fractions were identified by fluorescence detection of DiI and by phospholipid quantitation (Sigma). RNA sequencing on selected fractions was conducted by System Biosciences (CA). Proteomics on selected fractions was conducted by Bioproximity using LC-MS/MS (Chantilly, Va.). Pyruvate kinase protein and enzyme activity were assessed by ProteinSimple immunoassay and a colorimetric kinetic assay (Abcam, ab83432), respectively.

In Vitro Model: Smooth muscle cells (SMC) were treated with PBS or exosomes for 24 hours in either normoxia or hypoxia (4% oxygen). Experimental replicates were processed for microarray analysis using the Illumina platform (Qiagen) or for metabolomics analysis using the HD4 platform (Metabolon, RTP, N.C.). Oxygen consumption was measured using the abcam extracellular O2 consumption assay kit (Abcam, ab197243).

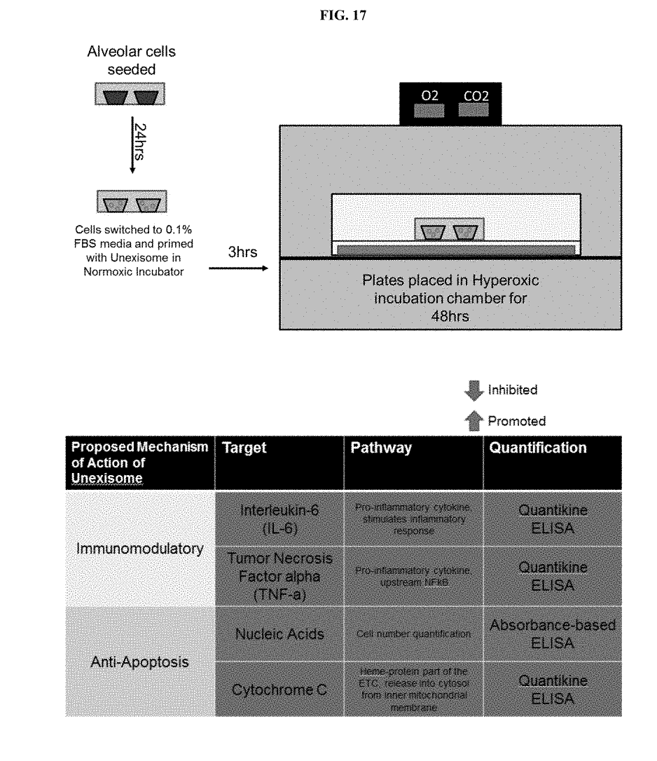

Alveolar cells were seeded for 24 hours, and switched to 0.1% FBS media and primed with a potent population of exosome in normoxic incubator for 3 hours, and plated in hyperoxic incubation chamber for 48 hours (FIG. 17).

SMC chronic hypoxia model: SMC are known to switch to a proliferative, non-apoptotic phenotype in PAH and hypoxia, leads to thickening of the vessels and arteries in the lungs, causing higher pressures and ultimately damages to the heart/negative symptoms in PAH. SMCs were cultured at normoxia, hypoxia (4% oxygen), and hypoxia (4% oxygen) with exosomes. During the culturing period, the cells were being treated twice a week for two weeks with potent exosomes. The resulting SMCs were analyzed by microarray gene expression (IPA) and/or global metabolomics.

### Example 9

### In Vitro Treatment with Unexisomes

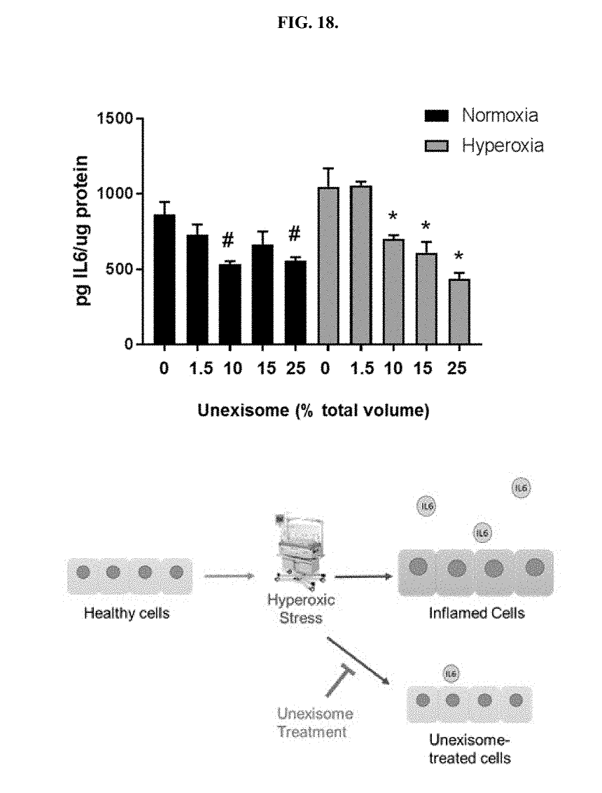

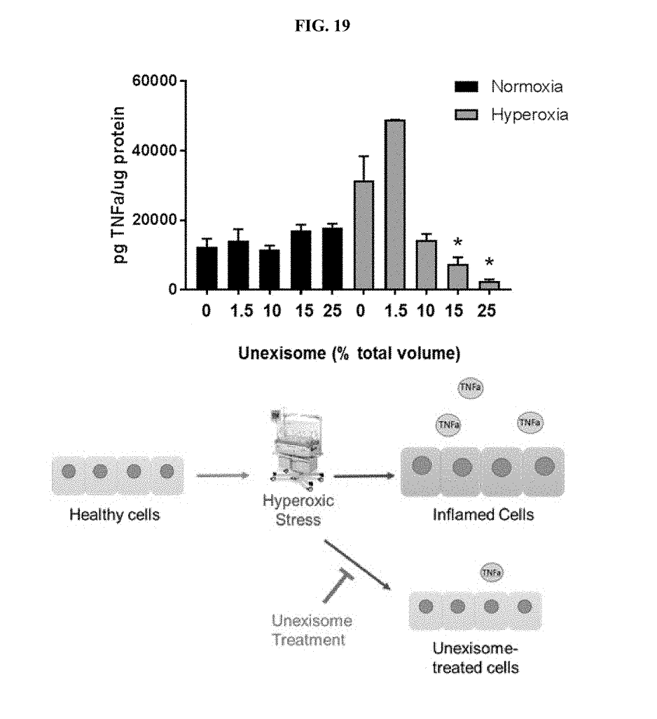

It has been demonstrated through in vitro experiments that unexisomes have immunomodulatory capacity. As shown in FIG. 18, unexisome treatment increased immunomodulatory capacity based on decreased IL-6 expression in cells exposed to hyperoxic stress (hyperoxia causes IL-6 release). As shown in FIG. 19, unexisome treatment increased immunomodulatory capacity based on reduced TNFα expression in cells exposed to hyperoxic stress (hyperoxia causes TNFα release).

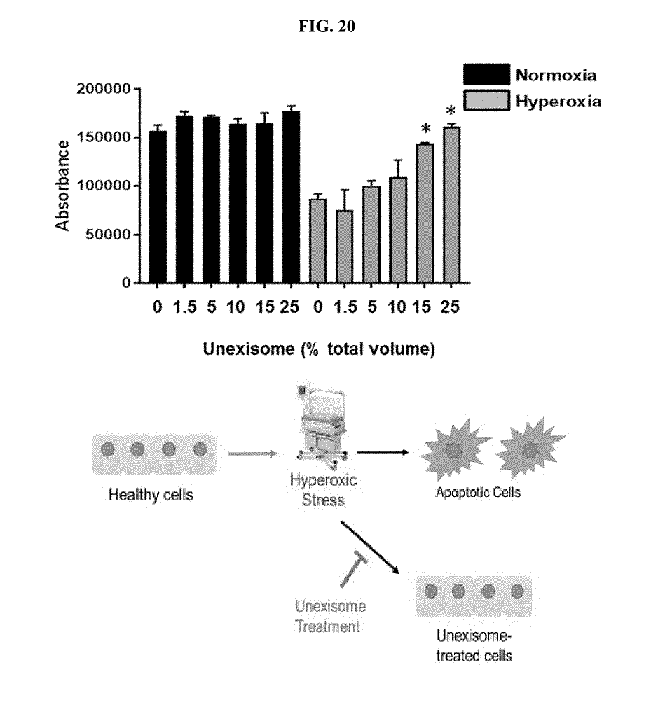

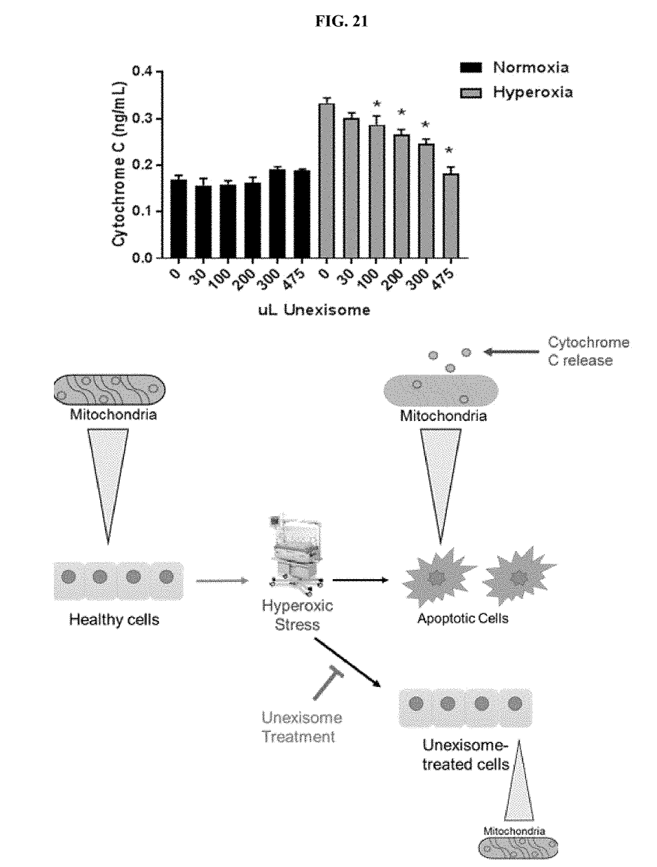

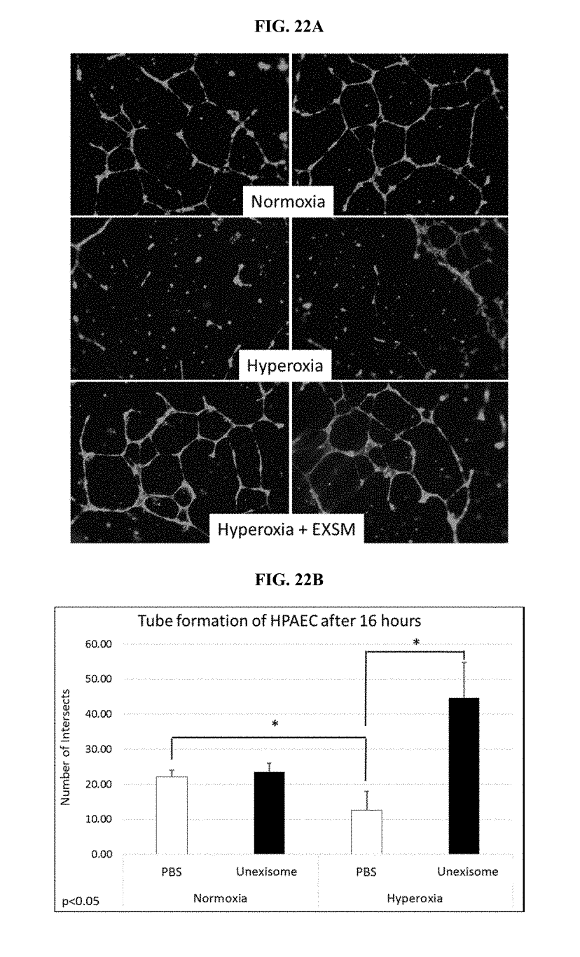

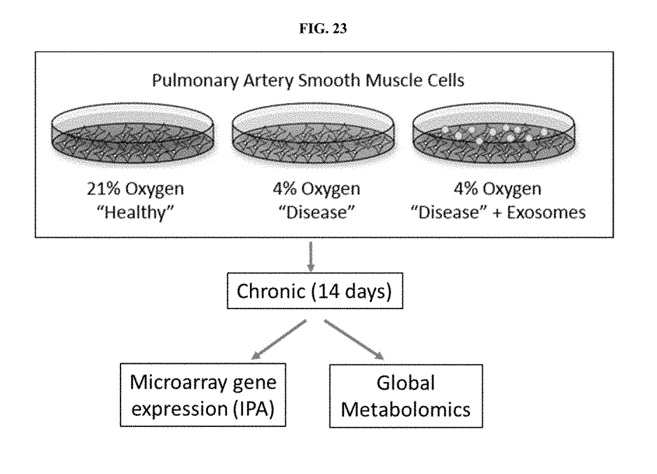

It has also been demonstrated through in vitro experiments that unexisomes have anti-apoptosis capacity. As shown in FIG. 20, unexisome treatment exhibited anti-apoptosis effect under hyperoxia, as indicated by increased absorbance, corresponding to increased number of cells. As shown in FIG. 21, unexisome treatment reduced cytochrome C release from cells exposed to hyperoxic stress.

It has been further demonstrated through in vitro experiments that unexisomes can be used to promote pulmonary angiogenesis in BPD. As shown in FIG. 22A, unexisome treatment can restore tube formation in acute hyperoxia exposure conditions.

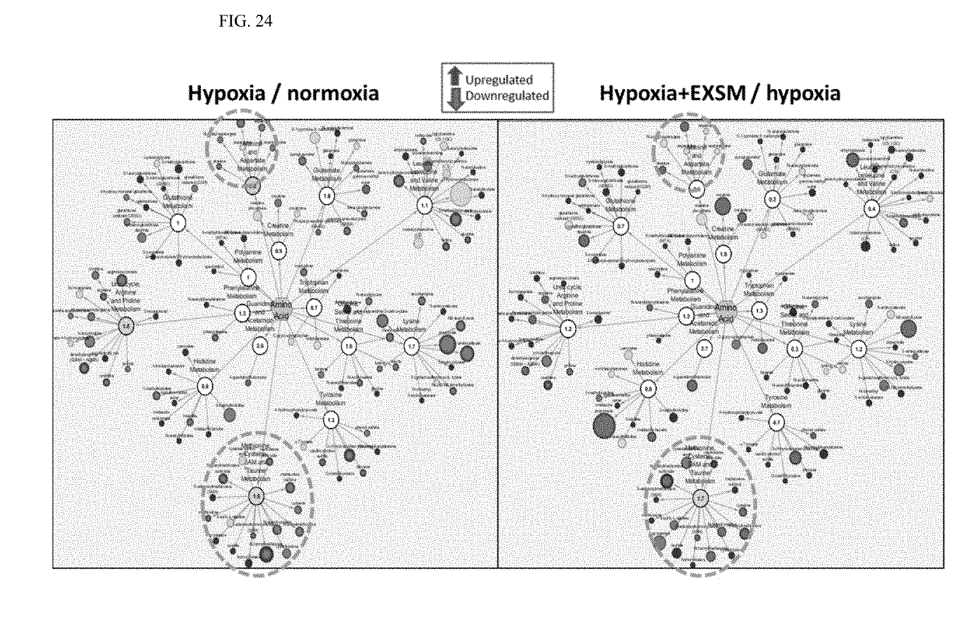

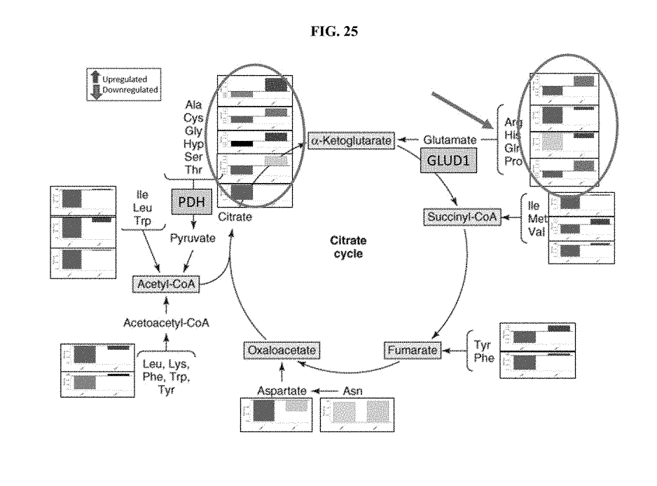

It has been additionally demonstrated through in vitro experiments that unexisomes can can be used to improve mitochondrial metabolism in BPD-associated PAH. As shown in FIG. 24, unexisomes upregulated amino acid metabolism in chronic hypoxia by global metabolite analysis of the intermediate metabolites within pathway. As shown in FIG. 25, unexisomes upregulated pyruvate and glutamate metabolism in chronic hypoxia.

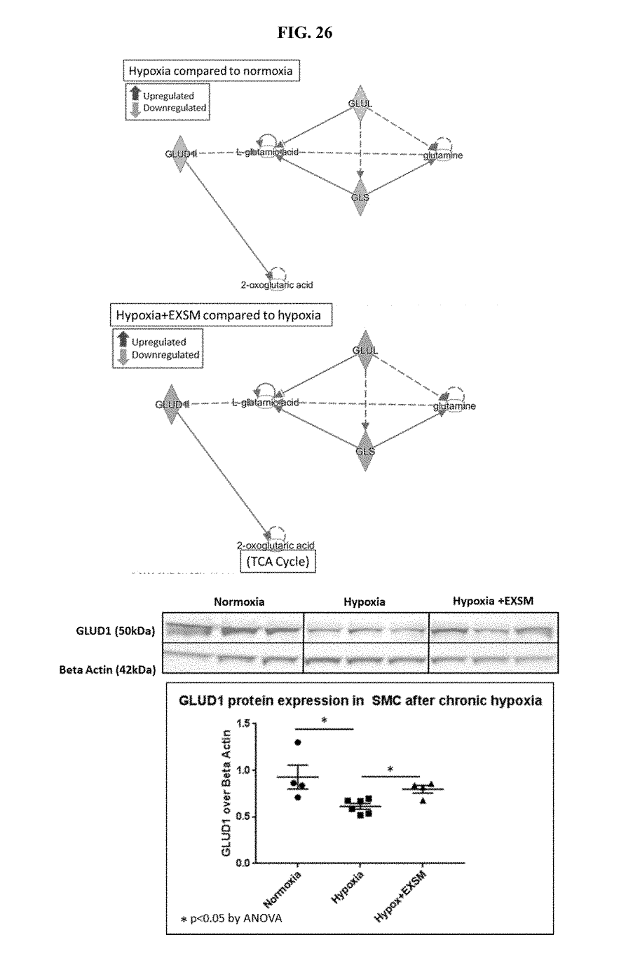

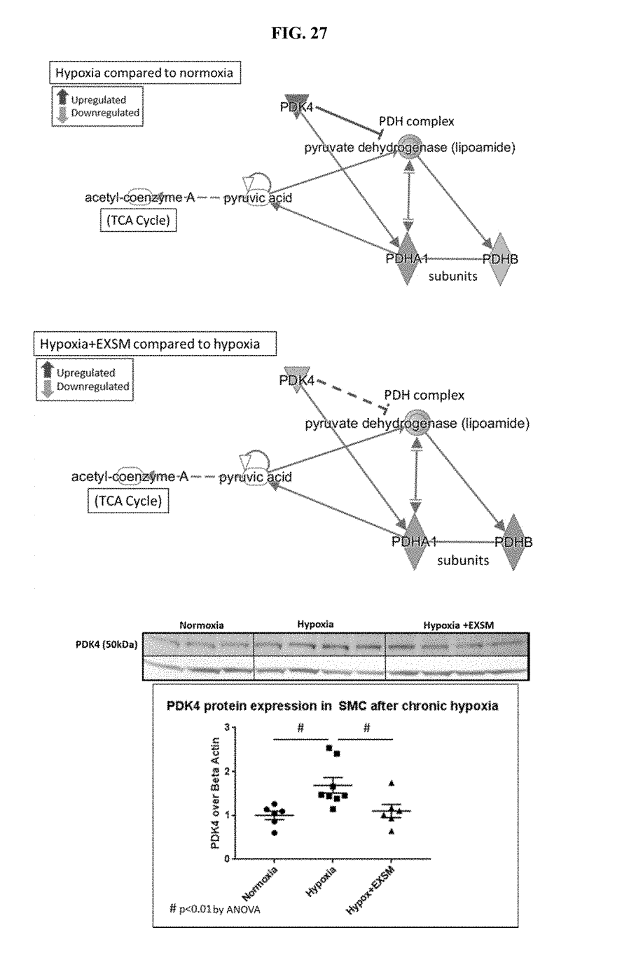

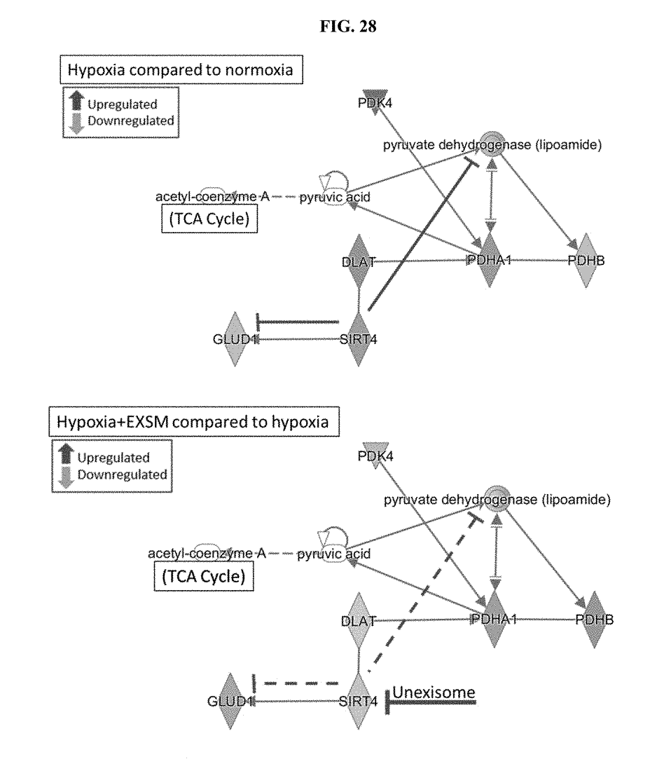

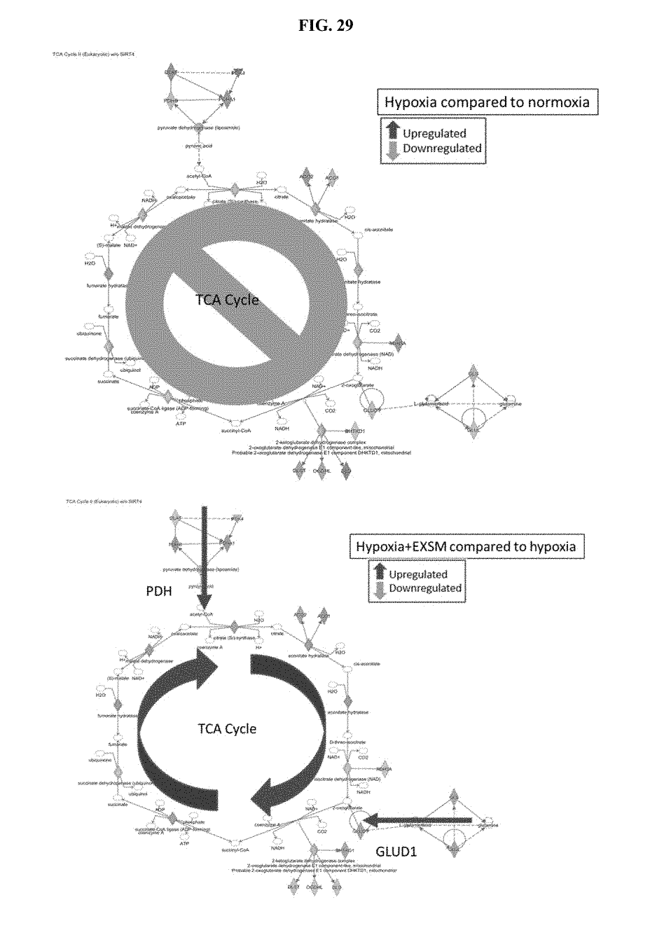

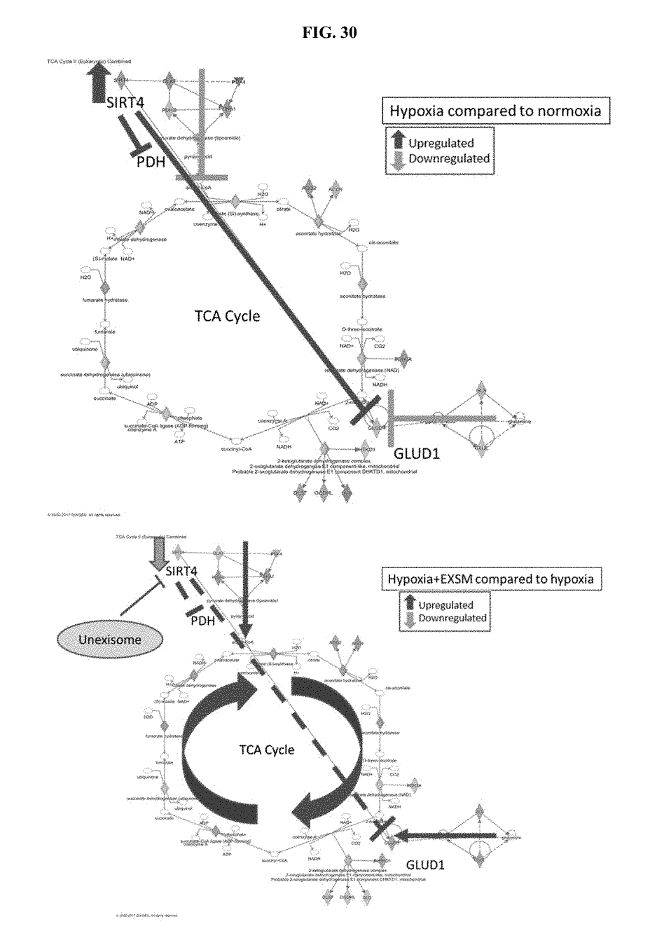

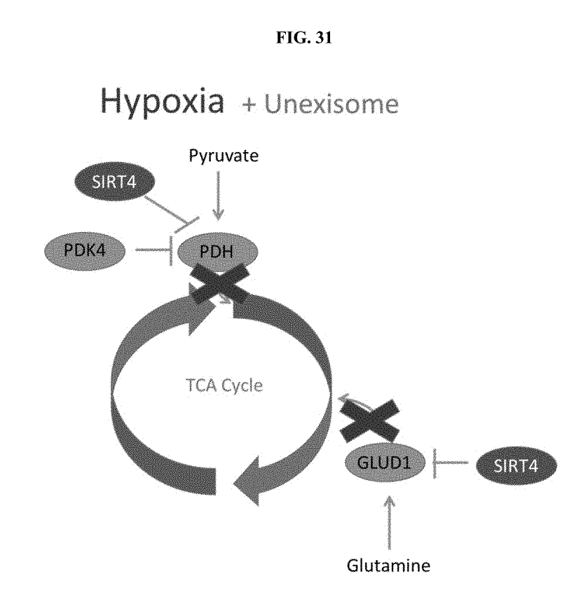

As shown in FIG. 26, unexisomes upregulated GLUD1 gene expression in chronic hypoxia. As shown in FIG. 27, unexisomes downregulated PDK4 in chronic hypoxia. As shown in FIG. 28, unexisomes downregulated SIRT4 in chronic hypoxia. SIRT4 gene inhibits 2 metabolic enzymes, GLUD1 and PDH, in an in vitro PAH model. SIRT4 gene was downregulated by unexisome treatment in vitro PAH model, while GLUD1 and PDH were upregulated by exosome treatment. SIRT4 is believed to be a target for exosome treatment. As shown in FIG. 29, unexisomes restored TCA cycle function by upregulating the downregulated genes in hypoxia (6 out of 9 enzymes in the TCA cycle are downregulated).

### Example 10

### In Vivo Treatment with Unexisomes

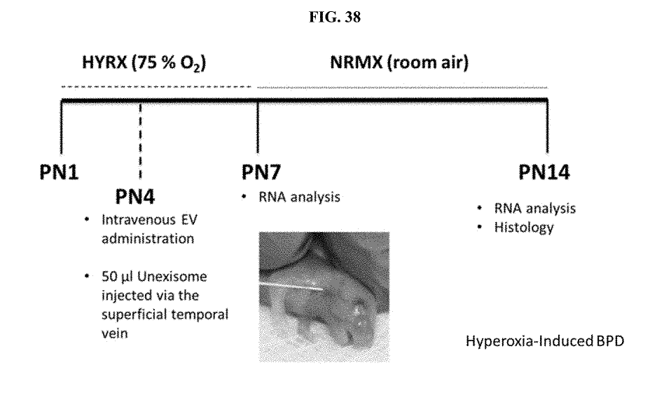

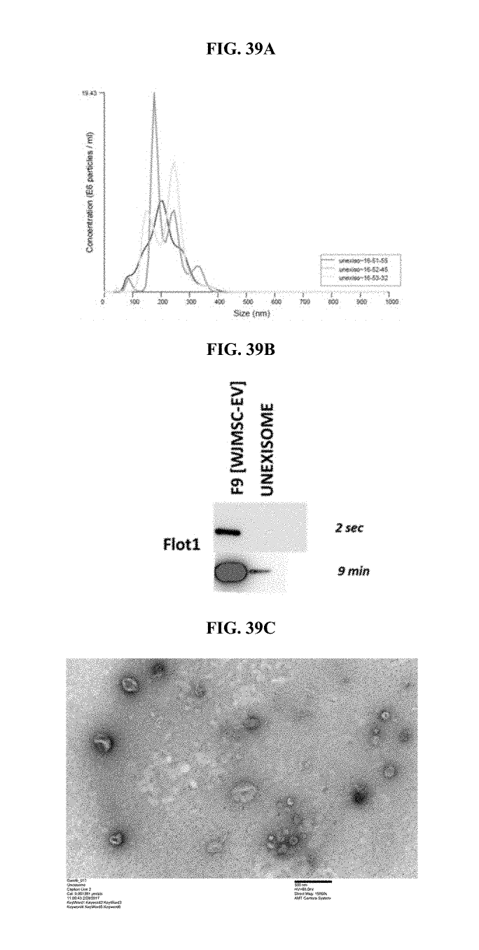

Hyperoxia-Induced BPD study: C57BL/6 mice were subject to 75% oxygen from day 1 to day 7 postnatal (PN), and switched to room air with normal oxygen level from day 7 to day 14 postnatal. At PN4, a single-dose of potent exosomes was injected into the superficial temporal vain. At PN7 and PN14, RNA and histology analysis were conducted (FIG. 38).

BPD induced PAH study: C57BL/6 mice were subject to 75% oxygen from day 1 to day 7 postnatal (PN), and switched to room air with normal oxygen level from day 7 to day 42 postnatal. At PN4, a single-dose of potent exosomes was injected into the superficial temporal vain. At PN7 and PN14, RNA, histology and cytometric analysis were conducted. At PN42, histology and cytometric analysis were conducted, and additionally physiological measurements, lung function tests, and RV pressure measurements were also conducted (FIG. 38).

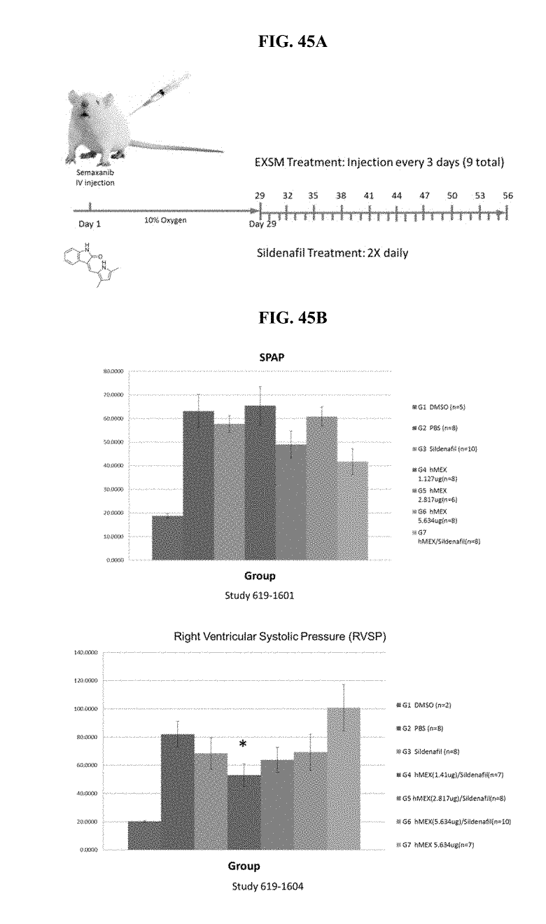

Combination treatment: C57BL/6 mice were subject to 10% oxygen from day 1 to day 29 and switched to normal condition from day 29 to day 56. On day 1, the mice were injected with semaxanib, and from day 29 to day 56, the mice were injected with sildenafil twice daily and with exosomes once every 3 days. RVSP level was measured (FIGS. 45A-45B).

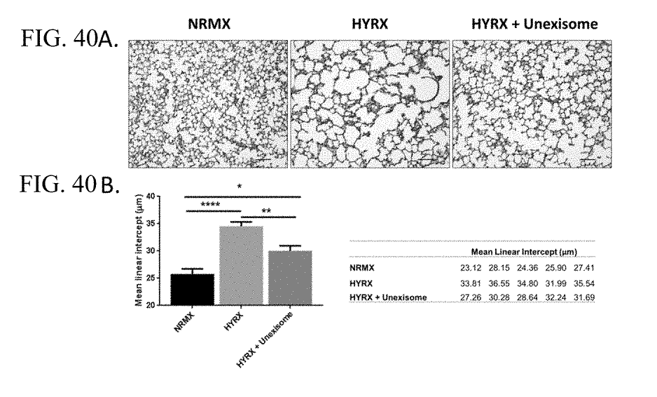

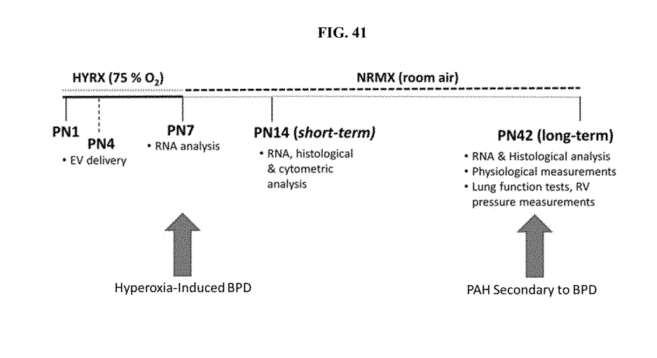

As shown in FIG. 40, unexisome treatment rescued BPD-associated alveolar simplification. FIG. 40A shows that treatment with potent exosomes rescues the hyperoxia (HYRX)-mediated increase in alveolar simplification compared to normoxia (NRMX). FIG. 40B shows quantification of mean liner intercept, which represents a surrogate of average air space diameter.

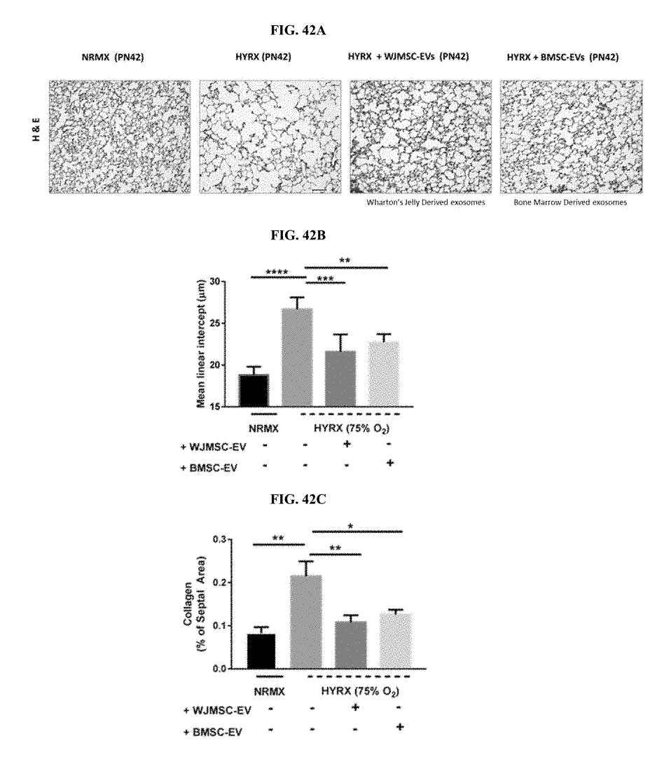

As shown in FIGS. 42A-42C, unexisome treatment rescued chronic alveolar simplification. FIG. 42A shows the images of cells under normoxia, hypoxia, and cells treated with Wharton's jelly derived exosomes, and bone marrow derived exosomes. FIG. 42B shows that exosomes rescued alveolar simplification in BPD-associated PAH mouse model. FIG. 42C shows that exosomes reduced modest lung fibrosis as shown by collagen deposition in the septal area in BPD-associated PAH mouse model.

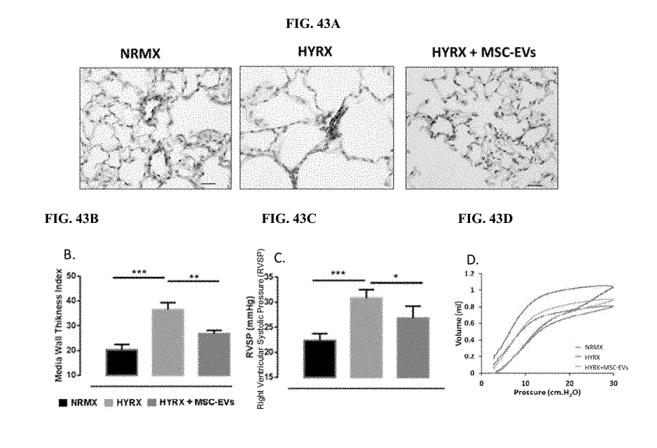

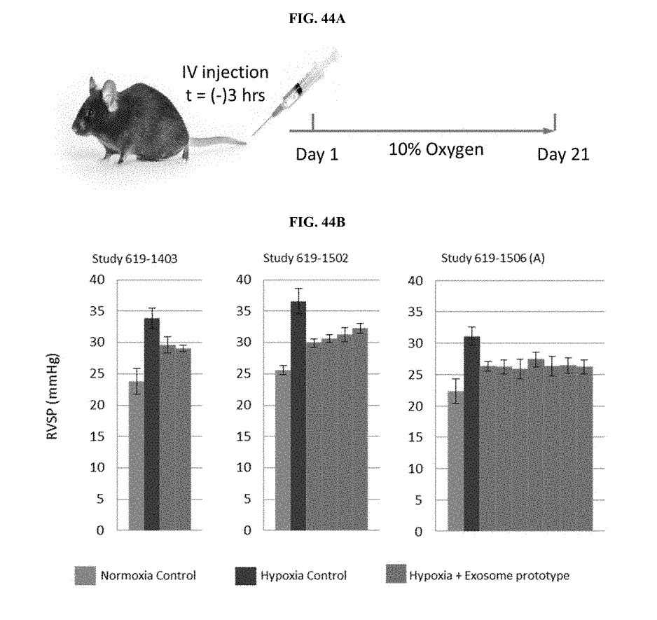

As shown in FIGS. 43A-43D, unexisome treatment rescued PAH pulmonary vascular remodeling demonstrated by a-smooth muscle actin stain (A and B) and pressure changes associated with PAH (C). PV-loops demonstrate a significant shift in hyperoxia (HYRX) mice, indicative of emphysema-like features of lung disease and air trapping when compared to normoxia (NRMX) controls. Exosomes showed a significant rescue in this shift, indicative of improved lung function.

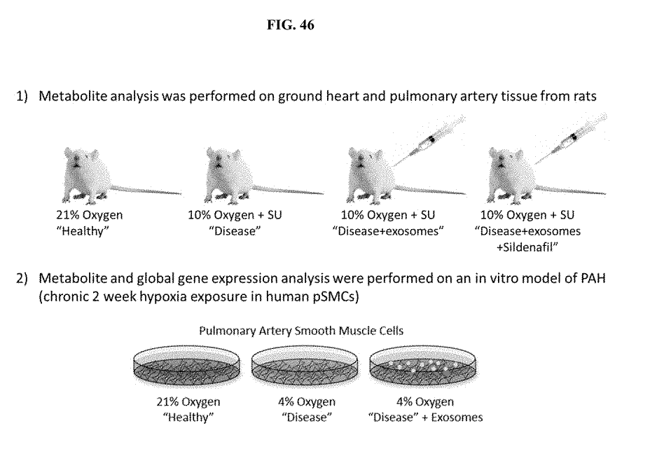

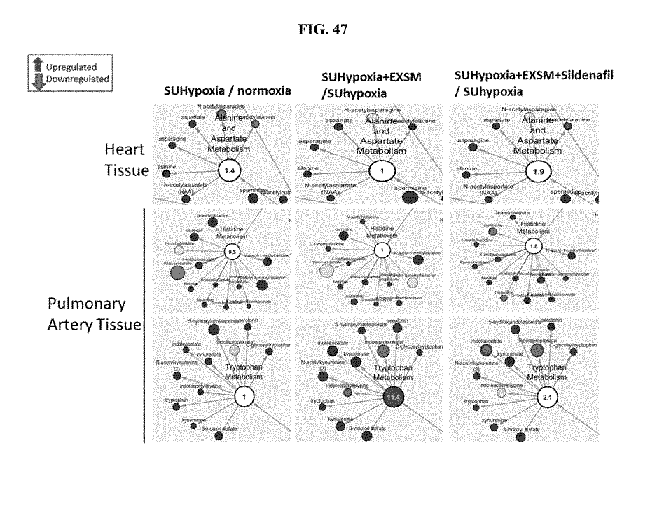

As shown in FIGS. 44A-44B, MSC exosomes prevented PAH in hypoxia-induced PAH mouse model. As shown in FIGS. 45A-45B, the combination therapy of exosome and sildenafil reversed PAH in Sugen/hypoxia-induced PAH mouse model. As shown in FIG. 46, exosomes upregulated amino acid metabolism in Sugen/hypoxia model of PAH.

### Example 11

### Characterization of Unexisomes

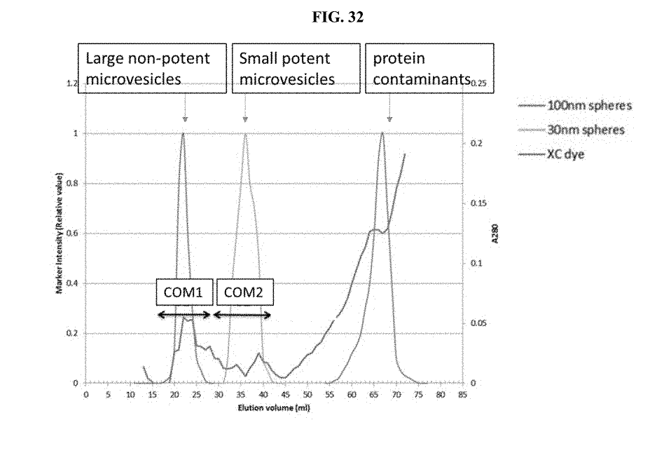

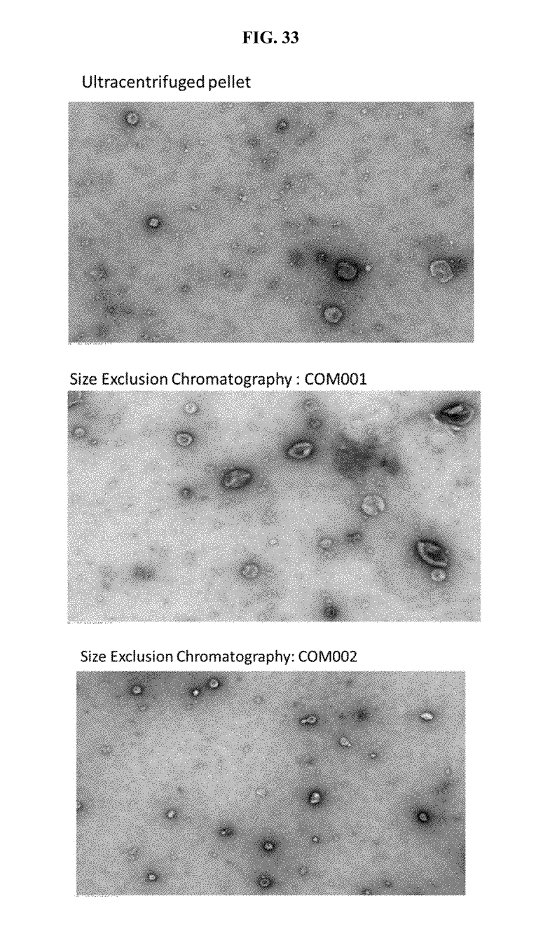

As shown in FIG. 32, isolation of the potent population of exosomes (COM2) using size exclusion chromatography result in significantly reduced contamination, compared to ultra-centrifugation or gradient separation which isolate not only exosomes of non-ideal size but also protein and non-potent microvesicles contaminants. As shown in FIG. 33, the isolated potent exosomes have more homogenous size and clearer image compared to ultracentrifuged samples.

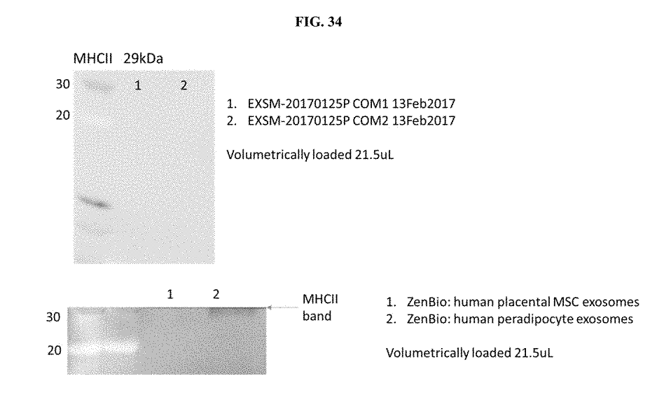

As shown in FIG. 34, the isolated potent exosomes are free of MHCII contamination. In contrast, commercially available exosomes from ZenBio contain MHCII contamination.

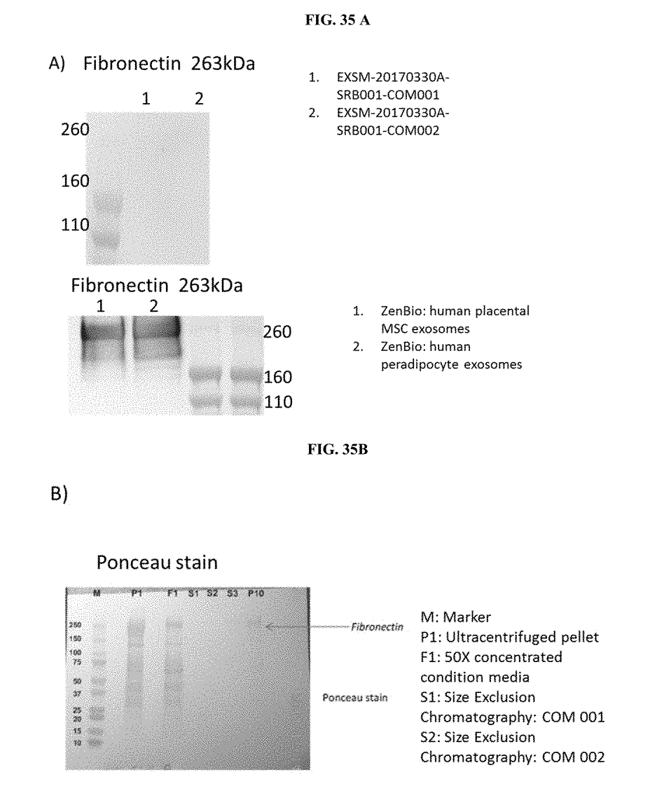

As shown in FIGS. 35A-35B, the isolated potent exosomes isolated by size exclusion chromatograph are free of fibronectin and other protein contaminations based on ponceau staining, while ZenBio commercially available ultracentrifuged preparation have fibronectin contamination.

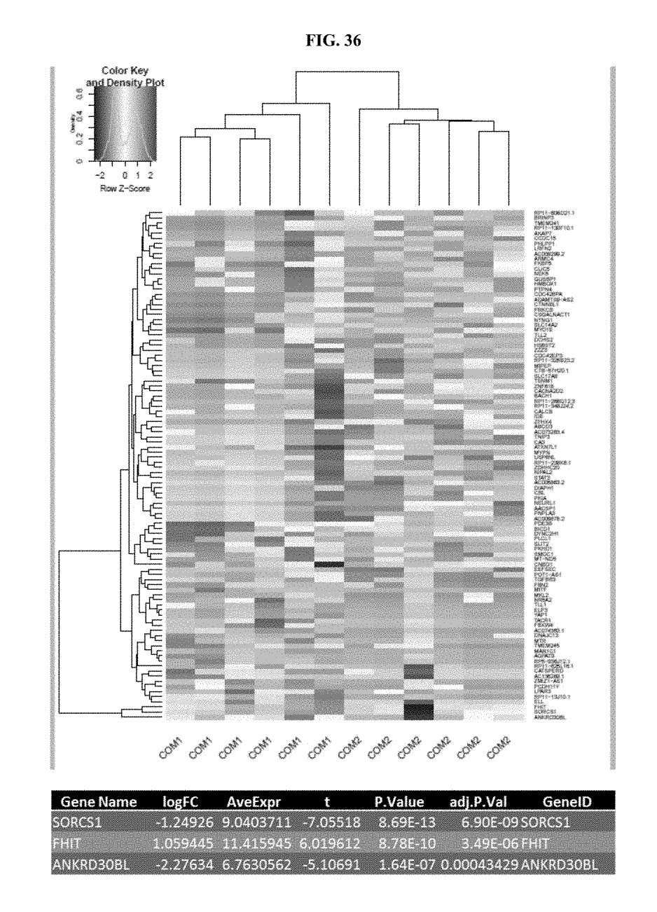

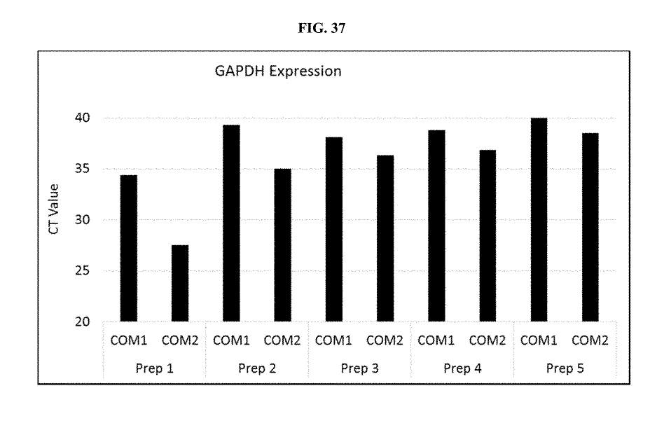

As shown in FIG. 36, in RNAseq clustering analysis of the potent population and contaminant microvesicles, the potent exosomes (COM2) are differentially clustering from contaminating microvesicles, and are enriched in SORCS1, FHIT and ANKRD30 BL. As shown in FIG. 37 the potent exosomes (COM2) have a lower CT value than COM1, corresponding to higher expression level of GAPDH expression.

1. A method of treating pulmonary hypertension, comprising administering to a subject in need thereof isolated extracellular vesicles or exosomes obtained from mesenchymal stromal cells, wherein the isolated extracellular vesicles or exosomes comprise extracellular vesicles or exosomes having increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells.

2. The method of claim 1, wherein the extracellular vesicles or exosomes comprise at least 20% more expression of the expression products compared to the average amount of the same expression product in all extracellular vesicles or exosomes obtained from the mesenchymal stromal cells.

3. The method of claim 1, wherein (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

4. The method of claim 1, wherein the gene is PK.

5. The method of claim 1, wherein the gene is ATPase.

6. The method of claim 1, wherein the isolated extracellular vesicles or exosomes normalize glucose oxidation in lung tissue of the subject.

7. The method of claim 1, wherein the isolated extracellular vesicles or exosomes have a PK activity of at least 0.15 nmol/min/mL.

8. The method of claim 1, wherein the isolated extracellular vesicles or exosomes are capable of reducing Right Ventricular Systolic Pressure (RVSP) of mice subjected to a three-week chronic hypoxia exposure by at least 10% compared to control mice subjected to a three-week chronic hypoxia exposure and treated with PBS.

9. The method of claim 1, wherein the isolated extracellular vesicles or exosomes are capable of increasing O₂ consumption by SMC cell lysates subjected to a 24-hour hypoxia exposure by at least 20% compared to control SMC cell lysates subjected to a 24-hour hypoxia exposure and treated with PBS control.

10. A method of treating a disease or condition associated with mitochondrial dysfunction, comprising administering to a subject in need thereof isolated extracellular vesicles or exosomes obtained from mesenchymal stromal cells, wherein the isolated extracellular vesicles or exosomes comprise extracellular vesicles or exosomes having increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells.

11. The method of claim 10, wherein (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

12. The method of claim 10, wherein the gene is PK.

13. The method of claim 13, wherein the isolated extracellular vesicles or exosomes a PK activity of at least 0.15 nmol/min/mL.

14. The method of claim 10, wherein the gene is ATPase.

15. The method of claim 10, wherein the isolated extracellular vesicles or exosomes normalize glucose oxidation in lung tissue of the subject.

16. The method of claim 10, wherein the disease or condition associated with mitochondrial dysfunction is associated with decreased mitochondrial glucose oxidation in the subject.

17. The method of claim 10, wherein the disease or condition associated with mitochondrial dysfunction is selected from the group consisting of Friedreich's ataxia, Leber's Hereditary Optic Neuropathy, Kearns-Sayre Syndrome, Mitochondrial Encephalomyopathy with Lactic Acidosis and Stroke-Like Episodes, Leigh syndrome, obesity, atherosclerosis, amyotrophic lateral sclerosis, Parkinson's Disease, cancer, heart failure, myocardial infarction (MI), Alzheimer's Disease, Huntington's Disease, schizophrenia, bipolar disorder, fragile X syndrome, and chronic fatigue syndrome.

18. A method of isolating extracellular vesicles or exosomes capable of treating or preventing pulmonary hypertension, comprising the following steps:
a. providing a culture media of mesenchymal stromal cells comprising extracellular vesicles or exosomes;
b. separating at least a portion of the extracellular vesicles or exosomes from the other components of the culture media;
c. isolating a extracellular vesicle or exosome population from other extracellular vesicle or exosome populations, wherein the population has increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells.

19. The method of claim 18, wherein the extracellular vesicle or exosome population is isolated by phospholipid detection.

20. The method of claim 18, wherein (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

21. The method of claim 18, wherein the gene is PK.

22. The method of claim 18, wherein the gene is APTase.

23. A method of isolating extracellular vesicles or exosomes capable of treating or preventing pulmonary hypertension, comprising the following steps:
a. providing a culture media of mesenchymal stromal cells comprising extracellular vesicles or exosomes;
b. separating at least a portion of the extracellular vesicles or exosomes from the other components of the culture media;
c. separating different populations of extracellular vesicles or exosomes based on molecular size;
d. treating hypoxia-exposed mice with the different populations of extracellular vesicles or exosomes;
e. measuring Right Ventricular Systolic Pressure (RVSP) of normoxia mice, hypoxia-exposed mice and hypoxia exposed mice treated with the extracellular vesicles or exosomes;
f. identifying a potent population of extracellular vesicles or exosomes based on the RVSP.

24. The method of claim 23, wherein a population of extracellular vesicles or exosomes is potent if the ratio of RVSP of hypoxia-exposed mice treated with the extracellular vesicles or exosomes to RVSP of hypoxia-exposed mice is 0.85 or less.

25. The method of claim 23, wherein a population of extracellular vesicles or exosomes is potent if delta RVSP is less than 5, wherein delta RVSP is RVSP of hypoxia-exposed mice treated with extracellular vesicles or exosomes minus RVSP of normoxia mice.

26. The method of claim 23, wherein in step c, different populations of extracellular vesicles or exosomes are separated by phospholipid detection.

27. The method of claim 23, wherein the potent population of extracellular vesicles or exosomes have increased expression of one or more expression products selected from the group consisting of (a) genes in the glycolysis pathway, (b) genes in the TCA cycle, and (c) genes in the electron transport chain as compared to the average amount of the expression products in all extracellular vesicles or exosomes obtained from the mesenchymal stromal cells.

28. The method of claim 23, wherein (a) the gene in the glycolysis pathway is selected from the group consisting of PK, AGI, ALDO, ALDOA, ENO3, GPI, HK2, HK3, PFK, PGM, TPI, GAPDH, ENO, and PGAM, (b) the gene in the TCA cycle is selected from the group consisting of MDH2, OGDH, PC, PDHA1, PDHB, SDHA, SDHC, and SUCLG2, and (c) the gene in the electron transport chain is selected from the group consisting of ETFA, ATPase, NDUFC2, NDUFB1,NDUF S5, NDUFA8, NDUFA9, NDUF S2, SDHA, SDHC, UQCRH1, Cox 6c1, and Cox10.

29. The method of claim 23, wherein the gene is PK.

30. The method of claim 23, wherein the gene is ATPase.

31. A method of isolating extracellular vesicles or exosomes capable of treating or preventing bronchopulmonary dysplasia, comprising the following steps:
a. providing a culture media of mesenchymal stromal cells comprising extracellular vesicles or exosomes;
b. separating at least a portion of the extracellular vesicles or exosomes from the other components of the culture media;
c. separating different populations of extracellular vesicles or exosomes based on molecular size;
d. treating hypoxia-exposed mice with the different populations of extracellular vesicles or exosomes;
e. measuring Right Ventricular Systolic Pressure (RVSP) of normoxia mice, hypoxia-exposed mice and hypoxia exposed mice treated with the extracellular vesicles or exosomes;
f. identifying a potent population of extracellular vesicles or exosomes based on the RVSP.

32. The method of claim 31, wherein the separating in step b is by size exclusion chromatography.

33. A composition comprising isolated extracellular vesicles or exosomes obtained according to claim 31.

34. The composition of claim 33, wherein the isolated extracellular vesicles or exosomes have a mean diameter of about 100 nm.

35. The composition of claim 33, wherein the isolated extracellular vesicles or exosomes express FLOT and/or ANXA2.

36. The composition of claim 33, wherein the isolated extracellular vesicles or exosomes have increased expression of mir204, compared to the average amount of mir204 in all extracellular vesicles or exosomes of the mesenchymal stromal cells.

37. The composition of claim 33, wherein the isolated extracellular vesicles or exosomes are secreted from MSCs containing increased expression of CD105, GAPDH, DLST, and/or ATP5A 1, compared to the average amount of CD105, GAPDH, DLST, and/or ATP5A1 in all the mesenchymal stromal cells.

38. The composition of claim 33, wherein the isolated extracellular vesicles or exosomes have increased RNA expression of SORCS1, FHIT and/or ANKRD30 BL, compared to the average amount of SORCS1, FHIT and/or ANKRD30 BL in all extracellular vesicles or exosomes of the mesenchymal stromal cells.

39. The composition of claim 33, wherein the isolated extracellular vesicles or exosomes are substantially free of MHCII contaminants.

40. The composition of claim 33, wherein the isolated extracellular vesicles or exosomes are substantially free of fibronectin.

41. A method of treating or preventing bronchopulmonary dysplasia, comprising administering to a subject in need thereof isolated extracellular vesicles or exosomes according to claim 31.

42. The method of claim 41, wherein the isolated extracellular vesicles or exosomes increase immunomodulatory capacity of the subject.

43. The method of claim 42, wherein the isolated extracellular vesicles or exosomes reduces IL-6 and/or TNFα expression in the subject.

44. The method of claim 41, wherein the isolated extracellular vesicles or exosomes promote angiogenesis of the subject.

45. The method of claim 44, wherein the isolated extracellular vesicles or exosomes reduce hyperoxia-induced apoptosis in the subject.

46. The method of claim 44, wherein the isolated extracellular vesicles or exosomes reduces Cytochrome C level in the subject.

47. The method of claim 41, wherein the isolated extracellular vesicles or exosomes increase mitochondrial metabolism of the subject.

48. The method of claim 41, wherein the isolated extracellular vesicles or exosomes restore tube formation in the subject.

49. The method of claim 41, wherein the isolated extracellular vesicles or exosomes upregulate GLUD1 and/or PDH gene expression in the subject.

50. The method of claim 41, wherein the isolated extracellular vesicles or exosomes downregulate PDK4 gene expression in the subject.

51. The method of claim 41, wherein the isolated extracellular vesicles or exosomes downregulate SIRT4 gene expression in the subject.

52. The method of claim 23, wherein the separating in step b is by size exclusion chromatography.

53. A composition comprising isolated extracellular vesicles or exosomes obtained according to claim 23.

54. The method of claim 1, further comprising administering sildenafil to the subject.

55. The method of claim 41, further comprising administering sildenafil to the subject.
